(19) United States
(12) Patent Application Publication
(10) Pub. No.: US 2016/0067301 A1
(43) Pub. Date: Mar. 10, 2016

(54) ANTAGONISM OF ABCG4, LYN KINASE, AND C-CBL E3 LIGASE TO INCREASE PLATELET COUNT AS THERAPY FOR THROMBOCYTOPENIA

(71) Applicants: Nan WANG, Flushing, NY (US); Alan R. TALL, Cresskill, NJ (US)

(72) Inventors: Nan WANG, Flushing, NY (US); Alan R. TALL, Cresskill, NJ (US)

(73) Assignee: The Trustees of Columbia University in the City of New York, New York, NY (US)

(21) Appl. No.: 14/783,789

(22) PCT Filed: Apr. 10, 2014
(86) PCT No.: PCT/US14/33659

§ 371 Date: Oct. 9, 2015

Related U.S. Application Data
(60) Provisional application No. 61/811,556, filed on Apr. 12, 2013.

(51) Int. Cl. A61K 38/05; A61K 31/4355; A61K 31/506; A61K 31/404; A61K 31/496; A61K 38/19

(52) U.S. Cl. CPC A61K 38/05; A61K 31/496; A61K 38/196; A61K 31/506; A61K 31/404; A61K 31/4355

(57) ABSTRACT

The present invention provides a method of treating a subject to increase the subject's platelet count which comprises administering to the subject an amount of one or more of an antagonist or inhibitor of ABCG4, Lyn kinase or c-CBL effective to antagonize or inhibit such ABCG4, Lyn kinase or c-CBL so as to thereby increase the subject's platelet count.

This application claims priority of U.S. Provisional Application No. 61/811,556, filed Apr. 12, 2013, the contents of which are hereby incorporated by reference.

This invention was made with government support under Grant number HL107653 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Blood is made up of three major cell types including red blood cells, white blood cells, and, platelets. Platlets are small, disk shaped clear cell fragments which are derived from fragmentation of precursor megakaryocytes. Platelets in the blood of mammals are involved in hemostasis, leading to the formation of blood clots. Platelets, or thrombocytes, are produced in the bone marrow and travel through blood vessels and stick together (clot) to stop any bleeding that occurs when blood vessels are damaged. If the number of platelets in the blood is too high, blood clots can form (thrombosis), which may obstruct blood vessels and result in such events as a stroke, myocardial infarction, pulmonary embolism or the blockage of blood vessels to other parts of the body, such as the extremities of the arms or legs. However, if the number of platelets in the blood is too low, excessive bleeding can occur.

Thrombocytopenia is a condition in which the body does not have a normal number of platelets in the blood (Erkurt, M. A. et al. 2012). The main symptom of thrombocytopenia is bleeding, either on the surface of the skin or internally. Various factors may interfere with the body's ability to make platelets. Causes of thrombocytopenia include bone marrow diseases such as leukemia, lymphoma, myelodysplastic syndrome or aplastic anemia; infectious diseases such as Epstein-Barr, cytomegalovirus, hepatitis, and HIV; autoimmune diseases such as immune thrombocytopenic purpura; an enlarged spleen which tends to trap platelets and prevent them from circulating in the bloodstream; chronic liver disease; HELLP syndrome; or megaloblastic anemia (Erkurt, M. A. et al. 2012). Thrombocytopenia may also be induced as a result of radiation or chemotherapy treatment; taking certain medications such as heparin; exposure to toxic chemicals; or drinking too much alcohol (Erkurt, M. A. et al. 2012).

Immune thrombocytopenic purpura (ITP) is a fairly common disorder (˜10 per 100,000 yearly) with a risk of bleeding but treatment options are limited. Current treatments include platelet infusion, steroids, splenectomy or infusions of NPlate® (romiplostim), which is a fusion protein analog of thrombopoietin (TPO) (Imbach, P. et al. 2011). Annual sales of romiplostim are about 500 million but effectiveness of the treatment is limited by side effects such as myalgia, joint and extremity discomfort, insomnia, thrombocytosis, and bone marrow fibrosis.

### SUMMARY OF THE INVENTION

The present invention provides a method of treating a subject to increase the subject's platelet count which comprises administering to the subject an amount of one or more of an antagonist or inhibitor of ABCG4, Lyn kinase or c-CBL effective to antagonize or inhibit such ABCG4, Lyn kinase or c-CBL so as to thereby increase the subject's platelet count.

### BRIEF DESCRIPTION OF THE FIGURES

FIG. 1. ABCG4 deficiency in bone marrow increases platelet count and accelerates atherosclerosis and thrombosis. Shown are results from Ldlr−/− mice transplanted with donor bone marrow cells from WT, Abcg4−/−, Abcg1−/− or Abca1−/− Abcg1−/− mice and fed a WTD diet for 12 weeks. (a) Quantification of proximal aortic root lesion area (with each symbol representing an individual mouse and the means of each group shown as horizontal lines) by morphometric analysis of H&E-stained sections. (b) Representative lacZ-stained proximal aortas from mice receiving Abca1−/− Abcg1−/− or Abcg4−/− bone marrow. Original magnification, ×40. Scale bars, 50 μm. (c) Platelet counts from Ldlr−/− mice receiving WT or Abcg4−/− bone marrow (BM). Data are shown as the means±s.e.m. (n=12 mice per group). (d) Cell surface expression of CD11b in platelet-associated Ly6-Chi monocytes or neutrophils in WTD-fed Ldlr−/− recipient mice. Anti-CD41, CD41-specific antibody; ISO, isotype-matching control antibody. (e,f) Concentrations of plasma platelet-derived microparticles (e) and percentages of reticulated platelets (f) in WTD-fed Ldlr−/− recipient mice. (g) Microthrombi formation on collagen in a flow chamber under shear flow using blood from WTD-fed Ldlr−/− recipient mice. Chamber surface coverage by the thrombi (fluorescence positive—light gray region) was quantified. (h) FeCl₃-induced carotid artery thrombosis in vivo in WTD-fed Ldlr−/− recipient mice. Data (d-h) are shown as the means±s.e.m. *P<0.05 for the comparisons between genotypes, ̂P<0.05 for basal compared to treatment. Statistical significance was determined by one-way analysis of variance (ANOVA) (a) or t test (c-h).

FIG. 2. Abcg4 is highly expressed in MkPs and regulates megakaryopoiesis and c-MPL expression. (a) Abcg4 mRNA expression in the indicated types of bone marrow and peripheral white blood cells in WT mice, as determined by quantitative real-time PCR (qPCR) (n=5 mice). GMP, granulocyte-macrophage progenitor; CLP, common lymphoid progenitor. (b) Flow cytometry analysis of CD41loCD71+ (ErP, erythrocyte progenitor), CD41+CD71lo (MkP) and CD41loCD71lo (MEP) cells from the parent MEP cell populations from WT and Abcg4−/− bone marrow shown in FIG. 9. (c) Abcg4 expression in MEPs and MkPs as assessed by qPCR. (d) ABCG4 protein in MkPs as assessed by immunofluorescence confocal microscopy. Cells were stained with isotype control antibody or antibody to ABCG4 (anti-ABCG4; green, unboxed regions) and DAPI (nuclei; blue, boxed regions). Scale bar, 5 μm. (e) Confocal microscopy of WT MkPs immunostained with antibodies to ABCG4 (green), 58K Golgi or TGN38 (red) and DAPI (blue). Scale bars, 5 μm. The arrows point to structures highly positive for ABCG4 or marker staining. (f,g) Quantification of the indicated bone marrow (BM) cell populations (f) and cell surface c-MPL expression (g) in Ldlr−/− recipient mice transplanted with WT or Abcg4−/− bone marrow and fed WTD for 12 weeks (n=5 mice per group). Also shown in g is a representative histogram for c-MPL expression. (h) Megakaryocyte-colony-forming unit MK-CFU) assay using hematopoietic progenitor cells (HPCs) harvested from WT and Abcg4−/− mice (n=5 mice per group). The micrographs show megakaryocyte colonies positively stained for acetylcholinesterase activity. Scale bars, 50 μm. (i) Platelet counts in WT and Abcg4−/− mice (n=5 mice per group) receiving a single dose of (50 μg per kg body weight) or vehicle control. Data (a,c,f-i) are shown as the means±s.e.m. *P<0.05 (t test) for WT compared to Abcg4+/−.

FIG. 3. ABCG4 deficiency decreases cholesterol efflux and increases membrane cholesterol content and proliferation of MkPs. (a) BODIPY-cholesterol efflux from WT and Abcg4−/− MkPs in response to treatment with cyclodextrin (CD; 2 mM) or rHDL (20 μg ml-1) for 2 h (n=4 samples per group). (b) BODIPY-cholesterol concentrations in WT and Abcg4−/− MkPs after cyclodextrin and BODIPY-cholesterol loading (n=4 samples per group). (c) Confocal fluorescence microscopy of MkPs from WT and Abcg4−/− mice incubated with cyclodextrin and BODIPY-cholesterol (green or light gray) and TO-PRO-3 (nuclei, blue or drak gray). Scale bars, 5 μm. (d) Confocal microscopy (left) and quantification (right) of WT and Abcg4−/− MkPs stained with filipin (red) and DAPI (blue). Scale bars, 10 μm. (e,f) EdU incorporation into (e) and cell surface c-MPL expression in (f) WT and Abcg4−/− MkPs as determined by flow cytometry after a 16-h treatment in the presence of TPO and with or without cyclodextrin (3 mM) or cyclodextrin-cholesterol complex (3 mM cyclodextrin) pretreatment for 30 min or rHDL (20 μg ml-1) for 16 h (n=4 samples per group). CD-chol, cyclodextrin-cholesterol complex. Data (a,b,d-f) are shown as the means±s.e.m. *P<0.05 (t test) for WT compared to Abcg4−/−, ̂P<0.05 (t test) for treatment effects.

FIG. 4. Increased MkP c-MPL expression and proliferation in ABCG4 deficiency involves altered activity of c-CBL and LYN. Shown are results for WT, Abcg4−/− and Lyn−/− MkPs (n=4 per group). (a) c-CBL phosphorylation in response to TPO quantified by phosphor-flow cytometry. Representative histograms are shown before and after 10 min of TPO treatment. p-c-CBL, phosphorylated c-CBL. (b) Cell surface c-MPL expression with or without MG132 treatment (10 μM) for 2 h in the presence of TPO. (c) c-CBL phosphorylation 5 min after TPO treatment with or without pretreatment with cyclodextrin (CD; 3 mM), cyclodextrin-cholesterol complex (CD-chol) (3 mM cyclodextrin) for 30 min or rHDL (20 μg apoA-I ml-1) for 2 h. (d) c-CBL phosphorylation with or without 5 mm of TPO treatment or SU6656 pretreatment (10 μg ml-1 for 2 h). (e) Cell surface c-MPL expression on MkPs with or without TPO or SU6656 treatment for 2 h. (f,g) Tyrosine-phosphorylated c-CBL (5 min in response to TPO; f) or cell surface c-MPL expression (2 h TPO treatment; g) with or without pretreatment with cyclodextrin-cholesterol complex (3 mM cyclodextrin) for 30 min. (h) Sixteen-hour EdU incorporation under the same conditions used in FIG. 3e,f. (i) Cell surface expression of c-MPL in bone marrow MkPs from WTD-fed BMT Ldlr−/− recipient mice transplanted with WT or Abcg4−/− bone marrow and treated with or without the LYN activator tolimidone (10 μM) in the presence of TPO for 2 h (n=4 samples per group). (j) Amounts of phosphorylated ERK1 and ERK2 (pERK1/2), phosphorylated AKT (pAKT) and phosphorylated STAT5 (pSTAT5) in WT or Abcg4−/− MkPs treated with or without TPO for 10 min. The dose of TPO was 30 ng ml-1 for all the assays shown. All data are shown as the means±s.e.m. *P<0.05 for WT compared to Abcg4−/− TPO treatment, ̂P<0.05 for treatment effects; NS, not significant. Statistical significance was determined by t test.

FIG. 5. rHDL suppresses platelet production in an ABCG4-dependent fashion in vivo. (a-c) Platelet counts determined by hematology analyzer (a) and the abundance of bone marrow MkPs (b) and bone marrow MkP c-MPL expression (c) determined by flow cytometry in WTD-fed Ldlr−/− recipient mice transplanted with WT or Abcg4−/− bone marrow 5 d after receiving a single infusion of vehicle or rHDL (100 mg apoA-I per kg body weight; n=5 mice per group). *P<0.05 (t test) for effect of rHDL infusion. (d) Platelet counts monitored weekly in WT mice transplanted with donor c-MplW515L-transduced bone marrow cells from WT (n=10) or Abcg4−/− (n=10) mice (n=5 mice per subgroup). The mice were given two infusions of rHDL (100 mg apoA-I per kg body weight) or vehicle, one at week 9 and one at week 10, as indicated. *P<0.05 for WT compared to Abcg4−/−, ̂P<0.05 for treatment effects. Statistical significance was determined by t test. (e,f) Platelet counts before (pre) and 5 d after (post) infusion (e) and total platelets after infusion (f) in patients with peripheral vascular disease who received a single infusion of rHDL (80 mg per kg body weight) or placebo. Data are presented as the mean decrease in total platelet numbers after infusion (n=7 per group) (f). *P<0.05 for placebo compared to rHDL; NS, not significant. Data (a-f) are shown as the mean±s.e.m. (g) Schematic model depicting the involvement of ABCG4 in the regulation of c-MPL expression in MkPs and MkP proliferation, leading to effects on platelet production, thrombosis and atherosclerosis.

FIG. 6. Atherosclerotic lesion and blood phenotype.

FIG. 7. Platelet cholesterol efflux.

FIG. 8. Flow cytometry overview.

FIG. 9. Expression of ABC transporters in hematopoietic cells.

FIG. 10. ABCG4 localization in MkPs.

FIG. 11. Platelet production pathway.

FIG. 12. Megakaryocyte quantification in the BM and spleen.

FIG. 13. Expression of cholesterol related genes.

FIG. 15. rHDL infusion decreases platelet production.

FIG. 16. ABCG4 deficiency leads to thrombocytosis and increases recovery of platelet count in ITP. (a) blood platelet count in irradiated Ldlr−/− mice receiving wild type or Abcg4−/− bone marrow. (b) Irradiated Ldlr−/− mice receiving wild type or Abcg4−/− bone marrow were injected with single dose of anti-CD41 antibody or isotype-matching control antibody (ISO). 24 hours after injection, the blood platelet count was determined. (c) Wild type or Abcg4−/− mice were injected a single dose of anti-CD41 antibody. The platelet count before (time 0) or after the injection was determined. *p<0.05.

FIG. 17. Abcg4−/− mice are more responsive to TPO induced platelet production. A single dose of TPO was injected into the wild type or Abcg4−/− mice. Platelet count was determined. *p<0.05.

FIG. 18. C-CBL inhibitor increases while LYN activator decreases cell surface c-MPL levels in MkPs. Bone marrow progenitor cells from WT or Abcg4^{-/-} mice were culture in the presence of TPO (10 ng/ml) with or without a Lyn kinase activator (Tolimidone, 10 μM) or a c-Cbl inhibitor (CRIN-1, 1 μM) for 2 hrs. MkPs were identified by cell surface markers and c-Mpl levels were detected via flow cytometry. *P<0.05 vs. WT basal.

### DETAILED DESCRIPTION OF THE INVENTION

The present invention provides a method of treating a subject to increase the subject's platelet count which comprises administering to the subject an amount of one or more of an antagonist or inhibitor of ABCG4, Lyn kinase or c-CBL effective to antagonize or inhibit such ABCG4, Lyn kinase or c-CBL so as to thereby increase the subject's platelet count.

In some embodiments, one of an antagonist or inhibitor of ABCG4, an antagonist or inhibitor of Lyn kinase or an antagonist or inhibitor of c-CBL is administered.

In some embodiments, an antagonist or inhibitor of ABCG4 is administered.

In some embodiments, an antagonist or inhibitor of Lyn kinase is administered.

5 In some embodiments, an antagonist Inhibitor of c-CBL is administered.

In some embodiments, two of an antagonist or inhibitor of ABCG4, an antagonist or inhibitor of Lyn kinase or an antagonist or inhibitor of c-CBL is administered.

In some embodiments, an antagonist or inhibitor of ABCG4 and an antagonist or inhibitor of Lyn kinase is administered.

In some embodiments, an antagonist or inhibitor of ABCG4 and an antagonist or inhibitor of c-CBL is administered.

In some embodiments, an antagonist or inhibitor of Lyn kinase and an antagonist or inhibitor of c-CBL is administered.

In some embodiments, an antagonist or inhibitor of ABCG4, an antagonist or inhibitor of Lyn kinase and an antagonist or inhibitor of c-CBL is administered.

In some embodiments, the Lyn kinase antagonist or inhibitor has the structure:

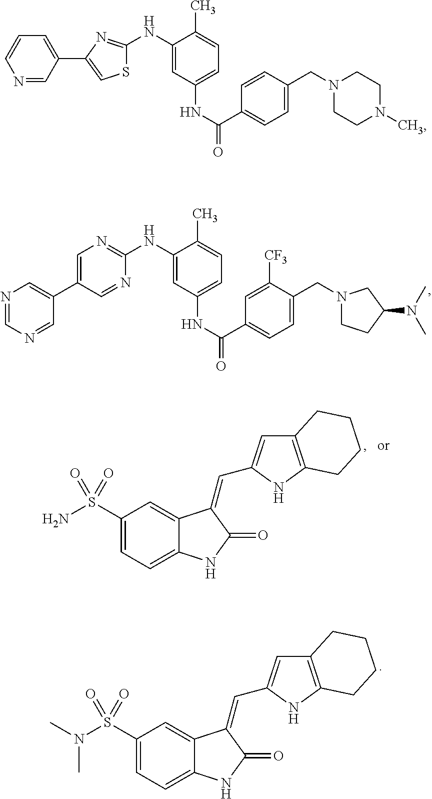

In some embodiments, the c-CBL antagonist or inhibitor has the structure:

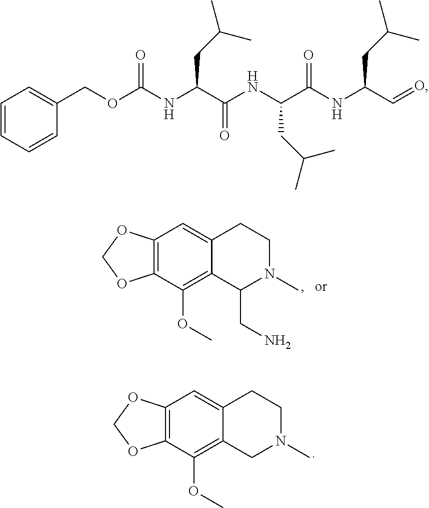 [SMILES: CC(C)C[C@@H](C=O)NC(=O)[C@H](CC(C)C)NC(=O)[C@H](CC(C)C)NC(=O)OCc1ccccc1 ; COc1c2c(cc3c1C(CN)N(C)CC3)OCO2 ; COc1c2c(cc3c1OCO3)CCN(C)C2]

In some embodiments, the method further comprises administering to the subject thrombopoietin.

In some embodiments, the method further comprises administering to the subject a thrombopoietin mimetic.

In some embodiments, the thrombopoietin mimetic is romiplostim.

In some embodiments, the thrombopoietin mimetic is eltrombopag.

In some embodiments, megakaryocyte production in the subject is increased.

In some embodiments, proliferation of megakaryocyte progenitor cells in the subject is increased.

In some embodiments, platelet production in the subject is increased.

In some embodiments, the subject is suffering from thrombocytopenia.

In some embodiments, the thrombocytopenia is idiopathic thrombocytopenia purpura.

In some embodiments, the thrombocytopenia is immune thrombocytopenia purpura.

In some embodiments, the thrombocytopenia is chemotherapy-induced thrombocytopenia.

In some embodiments, the thrombocytopenia is drug-induced thrombocytopenia.

In some embodiments, the subject is a human.

In some embodiments, the subject is a mammal.

As used herein, “thrombocytopenia” refers to any disease or disorder in which the blood has an abnormally low amount of platelets.

In some embodiments, the blood of the subject has a platelet count below 150,000 per μL.

In some embodiments, the blood of the subject has a platelet count below 50,000 per μL.

In some embodiments, the thrombocytopenia is caused or induced by bone marrow diseases such as leukemia, lymphoma, myelodysplastic syndrome or aplastic anemia; infectious diseases such as Epstein-Barr, cytomegalovirus, hepatitis, and HIV; autoimmune diseases such as immune thrombocytopenic purpura; an enlarged spleen which tends to trap platelets and prevent them from circulating in the bloodstream; chronic liver disease; HELLP syndrome; or megaloblastic anemia.

In some embodiments, the thrombocytopenia is caused or induced by radiation or chemotherapy treatment; taking certain medications such as heparin; exposure to toxic chemicals; or drinking too much alcohol.

“Idiopathic thrombocytopenia purpura” and “Immune thrombocytopenia purpura” are characterized by thrombocytopenia with normal bone marrow and the absence of other causes of thrombocytopenia.

The following are examples of ABCG4, Lyn kinase, or c-CBL inhibitors or antagonists; however, these are not the only ABCG4, Lyn kinase, or c-CBL inhibitors or antagonists that may be used in the method of the present invention. Various analogues of the below compounds, which are also ABCG4, Lyn kinase, or c-CBL inhibitors or antagonists, are used in the method of the present invention to increase platelet count in a subject. Other ABCG4, Lyn kinase, or c-CBL inhibitors or antagonists, which are structurally different from the below compounds, are used in the method of the present invention to increase platelet count in a subject.

The following compound is a Lyn kinase inhibitor or antagonist (Dubreuil, P. et al. 2009):

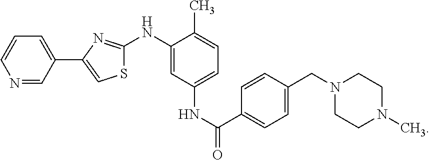 [SMILES: Cc1ccc(NC(=O)c2ccc(CN3CCN(C)CC3)cc2)cc1Nc1nc(-c2cccnc2)cs1]

The following compound is a Lyn inhibitor or antagonist (Santos, F. P. et al. 2010):

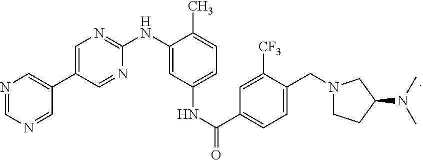 [SMILES: Cc1ccc(NC(=O)c2ccc(CN3CC[C@H](N(C)C)C3)c(C(F)(F)F)c2)cc1Nc1ncc(-c2cncnc2)cn1]

The following Lyn inhibitor or antagonist, SU6656, used in the method of the present invention may be purchased from Sigma-Aldrich, St. Louis, Mo., USA (Catalog No. S9692):

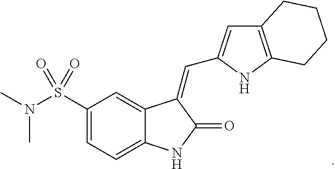 [SMILES: CN(C)S(=O)(=O)c1ccc2c(c1)/C(=C/c1cc3c([nH]1)CCCC3)C(=O)N2]

The following Lyn inhibitor or antagonist, SU6657 is related to SU6656 and has similar activity (Blake, R. A. et al. 2000):

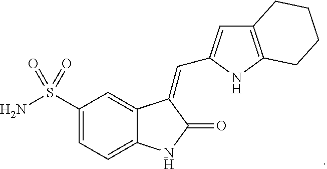 [SMILES: NS(=O)(=O)c1ccc2c(c1)/C(=C/c1cc3c([nH]1)CCCC3)C(=O)N2]

Additional Lyn kinase inhibitors VI201 and VI301 are available from Vassa Informatics (Kansas City, Mo., USA).

The following c-CBL inhibitor or antagonist, MG132, used in the method of the present invention may be purchased from Sigma-Aldrich, St. Louis, Mo., USA (Catalog No. C2211):

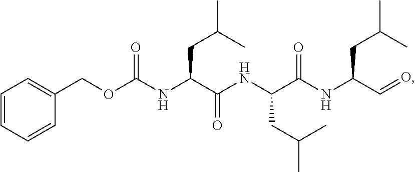 [SMILES: CC(C)C[C@@H](C=O)NC(=O)[C@H](CC(C)C)NC(=O)[C@H](CC(C)C)NC(=O)OCc1ccccc1]

The following c-CBL inhibitors or antagonists CRIN-1 and CRIN-2 are described in PCT international publication No. WO 2011/160016 A2, published Dec. 22, 2011:

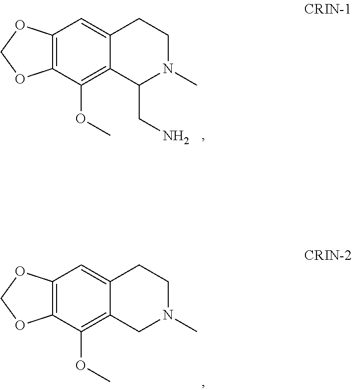 [SMILES: COc1c2c(cc3c1C(CN)N(C)CC3)OCO2 ; COc1c2c(cc3c1OCO3)CCN(C)C2]

The following are examples of thrombopoietin mimetics; however, these are not the only thrombopoietin mimetics that may be used in the method of present invention.

Romiplostim is a fusion protein analog of thrombopoietin, a hormone that regulates platelet production. The drug is marketed under the trade name NPlate®. Romiplostim is used to treat subjects with Immune Thrombocytopenia (Kuterm D. J. et al. 2010).

Eltrombopag is a compound that has been developed to treat thrombocytopenia (Jenkins, J M, et al. 2007). It is an agonist of the c-mpl (TpoR) receptor. Eltrombopag has the structure:

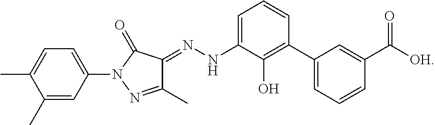 [SMILES: CC1=NN(c2ccc(C)c(C)c2)C(=O)/C1=N/Nc1cccc(-c2cccc(C(=O)O)c2)c1O]

The below structure, Tolimidone (also known as MLR 1023) may be purchased from Sigma-Aldrich, St. Louis, Mo., USA (MLR 1023: Catalog No. SML0371) or Activate Scientific, Germany (Catalog No. AS9568):

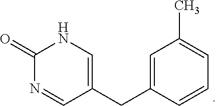

Except where otherwise specified, when the structure of a compound of the method of the present invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

The compounds used in the method of the present invention may be prepared by techniques well know in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain, the desired compounds.

The compounds used in the method of the present invention may be purchased from a chemical supplier, including Sigma-Aldrich, St. Louis, Mo., USA. However, this may not be the only means by which to synthesize or obtain the desired compounds.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30th edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” encompasses, e.g., inducing inhibition, regression, or stasis of a disease or disorder, or lessening, suppressing, inhibiting, reducing the severity of, eliminating or substantially eliminating, or ameliorating a symptom of the disease or disorder.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

used herein, a “pharmaceutically acceptable carrier” is pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylaspartamidephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Theta

### Abbreviations

ABC, ABC-binding cassette transporter; ACD, Atherosclerotic cardiovascular disease; BM, Bone marrow; BMT, bone marrow transplantation; CMP, Common myeloid progenitor cell; ET, Essential thrombocytosis; Gatal-HRD, Gatal-hematopoietic regulatory domain; GMP, Granulocyte/monocyte progenitor cell; HDL, High density lipoprotein; HSPCs, Hematopoietic stem and progenitor cell; ITP, Immune thrombocytopenia; JAK2, Janus kinase 2; Ldlr, Low density lipoprotein receptor; LSK, Lineage-negative (Lin−), Sca-1+, c-Kit+ cell; MEP, Megakaryocyte/erythrocyte progenitor cell, MF, Primary myelofibrosis; Mk, Megakaryocyte; MkP, Megakaryocyte progenitor cells; c-MPL, Thrombopoietin receptor; MPN, Myeloproliferative neoplasms; PF4, Platelet factor 4; PS, phosphatidylserine; rHDL, Cholesterol-poor apoA-I/phospholipid complex; q-PCR, Quantitative real-time RT-PCR; SKF, Src family kinase; SR-BI, Scavenger receptor class B member 1; TEG, Thrombelastography; TPO, Thrombopoietin; WTD, High fat hifh cholesterol diet; WT, Wild type.

### Materials and Methods

Mice and Treatments.

The Institutional Animal Care and Use Committee of Columbia University approved all the mouse studies. Abcg4−/−, Abcg1−/− Abca1−/− and Abcg1−/− mice in a C57BL/6J background were created as described and used in this study. Abcg4−/− mice were backcrossed onto C57BL/6J mice for more than ten generations. WT (C57BL/6J) and Ldlr−/− (B6.129S7-Ld1rtm1Her) mice were obtained from The Jackson Laboratory (Bar Harbor, Me.). For bone marrow transplantation studies, bone marrow from WT, Abcg4−/−, Abcg1−/− Abca1−/− or Abcg1−/− mice was transplanted into WT or Ldlr−/− recipient mice as described. For atherosclerosis studies, bone marrow-transplanted recipient mice were fed a Western diet (TD88137, Harlan Teklad) for the indicated period of time. Bone marrow-specific retro-viral expression of murine c-MPL^{W515L} was established as described44 using WT C57BL/6J mice as the recipient and WT C57BL/6J or Abcg4−/− mice as the bone marrow donor. Where indicated, vehicle (saline), rHDL or TPO (R&D Systems) was injected at the indicted dose into the mice through the tail vein. rHDL (CSL-111) was provided by CSL Behring AG, Bern, Switzerland; CSL-111 is composed of human apoA-I and phosphatidylcholine from soy bean in a ratio of 1:150. All patients gave their informed consent to the study, which was approved by the Human Ethics Committee of the Alfred Hospital and conducted in accordance with the principles of the Declaration of Helsinki 2000.

Femurs and tibia of Lyn−/− mice used to prepare Lyn−/− bone marrow cells were kindly provided by A. L. DeFranco of the University of California, San Francisco. The mice were created as described (Chan, V. W. et al. 1997) and backcrossed at least 15 generations onto the C57BL/6 background.

MG132 (474790) and SU6656 (572635) were from EMD Millipore (Darmstadt, Germany). These compounds were dissolved in DMSO as 10 mM (MG132) or 10 mg/ml (SU6656) stocks and diluted to the indicated concentrations in cell culture medium.

Histochemistry.

Tissues and proximal aortas were serially paraffin sectioned and stained with H&E for morphological analysis as described. The aortic lesion size of each mouse was calculated as the mean of the lesion areas in five aortic sections. Bone samples were decalcified with EDTA solution before cryosectioning. Antibody to von Willibrand factor (Dako, A0082, 1:500 dilution) was used to stain megakaryocytes in bone and spleen sections. lacZ staining of frozen sections of mouse bone, spleen or proximal aorta was carried out using a β-Galactosidase Staining Kit (Cell Signaling Technology, Danvers, Mass.).

Complete Blood Count.

Complete blood counts were quantified using whole blood collected from tail bleeding. A FORCYTE Veterinary Hematology Analyzer (Oxford Science, Inc.) was used for the analysis.

Plasma and Cellular Lipids.

Plasma lipoprotein cholesterol and triglyceride concentrations were determined by colorimetric enzymatic assays using assay kits from Wako Diagnostics (Japan). Platelets were isolated from platelet-rich plasma, which was prepared from a low-speed spin of EDTA-treated mouse plasma, and platelet cholesterol content was measured by gas chromatography after lipid extraction.

Cholesterol Efflux.

For platelet cholesterol efflux studies, platelets were isolated from platelet-rich plasma by centrifugation at ˜3,500 r.p.m. for 10 min in an Eppendorf centrifuge. Platelet-rich plasma was prepared from a low-speed spin (300 g for 7 min) of mouse plasma in the presence of 5 mM EDTA. The isolated platelets were resuspended in DMEM cell culture medium plus 0.2% BSA. Cyclodexrin-cholesterol complexes containing [3H]cholesterol were prepared as described and added to final concentrations of ˜3 mM cyclodextrin and ˜1 μCi [3H]cholesterol ml⁻¹, and the mixture was incubated at 37° C. for 30 min. The labeled platelets were then washed three times with the same medium by a brief spin at 300 g for 5 min and resuspension. rHDL was then added to initiate cholesterol efflux, which was allowed to proceed for the indicated time period. Cholesterol efflux was determined as a percentage efflux: (count of supernatant/total count)×100.

To measure cholesterol efflux from MkPs to HDL, total bone marrow cells were labeled by incubation with 0.03 mM methyl-β-cyclodextrin and BODIPY-cholesterol (molar ratio cyclodextrin:cholesterol:BODIPY-cholesterol of 40:0.8:0.2; Avanti Polar Lipids, Alabama, USA) in Iscove's Modified Dulbecco's Medium (IMDM) plus 0.2% BSA at 37° C., 5% CO₂ for 30 min. The cells were washed three times with fresh IMDM by a brief spin at 800 g for 2 min and resuspension in the same medium. Cyclodextrin or rHDL was then added to the cell suspension at the indicated concentration to initiate cholesterol efflux for the indicated periods of time. Efflux was stopped by a brief spin in a microcentrifuge and removal of the acceptors. Samples treated without cyclodextrin or rHDL were used as the baseline for efflux. To assess BODIPY-cholesterol content in MkPs, the cell suspension was stained with a cocktail of lineage markers (cell surface antigen 1 (Sca1), CD127, CD45R, CD19, CD11b, CD3e, TER-119, CD2, CD8, CD4 and Ly-6C/G, all APC conjugated; all from eBioscience) and progenitor cell markers, c-Kit and CD41 (eBioscience), CD16 and CD32 (FcγRII/III) and CD34 (BD Biosciences). All antibodies were used at 1:200 dilution. MkPs were identified as lineage (Lin)⁻, c-Kit⁺, CD16/CD32^{lo}, CD34^{lo} and CD41⁺, and the MFI of BODIPY-cholesterol from MkPs was measured by flow cytometry (LSRII, BD Biosciences) to assess BODIPY-cholesterol content in MkPs or cholesterol efflux: (1−remaining MFI/baseline MFI)×100.

Flow Cytometry-Based Proliferation Studies.

Blood leukocytes and bone marrow HSPCs were stained and analyzed or sorted as described49. Briefly, bone marrow cells from mouse femurs and tibias were stained with a cocktail of antibodies to lineage-committed cells (CD45R, CD19, CD11b, CD3e, TER-119, CD2, CD8, CD4 and Ly-6C/G, all FITC conjugated; eBioscience), with antibodies to Sca1 (Biolegend) and c-Kit (eBioscience) to identify HSPC populations and LSK (Lin-Sca1+c-Kit+) cells and with antibodies to CD16/CD32 (FcγRII/III) and CD34 (BD Biosciences) to separate CMP (Lin-Sca1-c-Kit⁺CD34^{int}FcγRII/III^{int}), GMP (Lin⁻Sca1^{˜}c-Kit⁺CD34^{int}FcγRII/III^{hi}) and MEP (Lin⁻Sca1⁻c-Kit⁺CD34^{lo}FcγRII/III^{lo}) cell populations. All antibodies were used at 1:200 dilution, For DNA content analysis (G2M phase), bone marrow cells were fixed and stained with DAPI (Invitrogen) before flow cytometry analysis. To determine in vivo cell proliferation, EdU (Invitrogen; 1 mg per mouse) was injected into mice through the tail vein 24 h before the mice were euthanized. Cells were immunostained as described above in preparation for flow cytometry. Cells were then fixed and permeabilized using 0.01% saponin (wt/vol; Fluka) and 1% FCS (vol/vol) in IC fixation buffer (eBiosciences) for 30 min. Cells were then washed and stained with Alexa Fluor-conjugated azides using the Click-iT system (Invitrogen). Proliferation was quantified as the percentage of EdU⁺cells by flow cytometry.

Quantification of Reticulated Platelets.

Undiluted EDTA-anticoagulated blood (5 μl) was mixed with a phycoerythrin (PE)-conjugated antibody to CD41 and the fluorescent DNA dye thiazole orange (final concentration 1 μg ml⁻¹) and incubated at room temperature for 20 min. Samples were then fixed by adding 1 ml of 1% formaldehyde in PBS. Data acquisition using logarithmic amplification of lightscatter and fluorescence signals was performed. PE-positive cells were gated in a thiazole orange versus PE dot plot.

Real-Time qPCR.

RNA extraction, complementary DNA synthesis and qPCR of HSPCs were performed as described (Murphy, A. J. et al. 2011). The quality of RNA samples was determined using agilent 2100 Bioanalyzer and an RNA 6000 LabChip. The primer sequences used for qPCR are shown in the below table:

| Gene | Forward primer | Reverse primer |
|---|---|---|
| Abca1 | CAGCTTCCATCCTC | CCACATCCACAACT |
| | CTTGTC | GTCTGG |
| Abcg1 | GTACCATGACATCG | AGCCGTAGATGGAC |
| | CTGGTG | AGGATG |
| Abcg4 | CGTGCTCACCTTTC | CGATGCTGCAGTAC |
| | CCTTAG | ACCACT |
| Hmgcs1 | GCCGTGAACTGGGT | GCATATATAGCAAT |
| | CGAA | GTCTCCTGCAA |
| Ldlr | GAGGAACTGGCGGC | GTGCTGGATGGGGA |
| | TGAA | GGTCT |
| Scarb1 | TCCCCATGAACTGT | GTTTGCCCGATGCC |
| | TCTGTGAA | CTTGA |
| β-actin | AGCCATGTACGTAG | GTGGTGGTGAAGCT |
| | CCATCC | GTAGCC |
| 36B4 | CCTGAAGTGCTCGA | CCACAGACAATGCC |
| | CATCAC | AGGAC |

MR-CET Assay.

Primary bone marrow HSPCs obtained by FACS were plated in methylcellulose-based medium (5,000 cells per assay) containing TPO (50 ng interleukin-6 (IL-6) (20 ng ml⁻¹) and IL-3 (10 ng ml⁻¹) and incubated for 8 d according to the manufacturer's protocol (Megacult-C, Stemcell Technologies). Cultures were fixed, and megakaryocyte colonies were visualized by staining for acetylcholinesterase activity. Nuclei were counterstained with Harris' hematoxylin. Colonies containing more than three megakaryocytes were scored as MK-CFUs.

ABCG4-Specific Antibody.

The rabbit antibody to ABCG4 was custom made by Pacific Immunology (CA, USA) against a synthetic ABCG4 peptide (KKVENHITEAQRFSHLPKR). Monospecific anti-peptide antibodies were purified using a peptide-affinity column. The specificity of the antibody for ABCG4 protein was assessed by immunofluorescence microscopy, which showed specific immunofluorescence signals in HEK293 cells expressing ABCG4 but not HEK293 cells transfected with mock vectors. Rabbit polyclonal antibody to c-MPL was used and the specificity of the antibody against cell surface c-MPL in flow cytometry has been reported previously (Tong, W. et al. 2007; Bersenev, A. et al. 2008).

Neutrophil and Monocyte Platelet Aggregates.

Blood was collected through the tail vein into EDTA-lined tubes on ice to prevent leukocyte activation. Red blood cells (RBCs) were lysed, and the washed cells were then stained with CD45 (Invitrogen), CD115 (eBioscience), Gr1 (Ly6-C/G; BD Biosciences), CD11b (eBioscience) and CD41 (eBioscience) at 1:200 dilution for 30 min on ice. The cells were carefully washed, resuspended in FACS buffer and run on an LSRII flow cytometer to detect leukocyte platelet interactions and leukocyte activation. Viable cells were selected on the basis of forward and side scatter characteristics, and then CD45+ leukocytes were selected. Ly6-C^{hi} monocyte platelet aggregates were identified as CD115⁺Gr1^{hi} (Ly6-C^{hi}) and CD41⁺. Neutrophil-platelet aggregates were identified as CD115-Gr1+ (Ly6-G+) and CD41+. Platelet-dependent activation of Ly6-Chi monocytes and neutrophils was measured as CD11b MFI after subtracting the expression of CD11b on Ly6-C^{hi} or neutrophils, which stained negative for platelets (CD41⁻).

Platelet-Derived Microparticles.

Equal amounts of mouse plasma (20 μl) were diluted with 4-(2-hydroxyethyl)-1-piperazineethanesulfonic acid (HEPES) binding buffer (80 μl) and then incubated with annexin V and antibody to CD41. Equal amounts of 1-μm beads (Invitrogen) were added to the sample as a size standard, which was then run on an LSRII flow cytometer. Platelet-derived microparticles were detected as particles less than 1 μm in size that stained positive for CD41 and annexin V. A standard amount of beads was acquired to ensure accurate counting in each sample. Data were converted to the number of microparticles per 1 μl of whole blood.

FeCl₃-Induced Carotid Artery Thrombosis.

Mice were anesthetized, and a cervical incision was made to expose the common carotid artery. A miniature Doppler flow probe (TS420 transit-time perivascular flow meter, Transonic Systems Inc.) was placed on the carotid artery to monitor blood flow. The injury to the artery was induced by a piece of Whatman paper (2 mm×2 mm) saturated with 5% FeCl₃. The time until the cessation of the blood flow was recorded as the occlusion time.

Ex Vivo Flow Chamber Assay.

Heparin (5 U ml⁻¹)-anticoagulated whole blood was incubated with 1 μM of the fluorescent dye DiOC6 (Sigma, St. Louis. Mo., USA) for 10 min at 37° C. The fluorescently labeled whole blood was then perfused over a collagen-coated glass cover surface (microcapillary glass tube coated with 100 μm ml-1 Horm collagen (Nycomed) overnight) at a controlled shear rate (1,800 using a syringe pump for 3 min. Adherent platelets and aggregates in the chamber were washed and examined under an inverted fluorescent microscope, and micrographs of adhered platelets were recorded for analysis. Flow chamber surface coverage by the thrombi was calculated using Imaged.

c-MPL Expression.

After harvesting bone marrow progenitor cells, RBCs were lysed, and the cells were resuspended in FACS buffer, Bone marrow cells were stained for MkPs as stated above, and c-MPL or isotype control antibodies were included. Cells were then washed and stained with a fluorescently conjugated secondary rabbit-specific antibody to detect the antibody to c-MPL for a further 30 min on ice. Following this, the cells were washed, resuspended in FACS buffer and run on an LSRII flow cytometer. MEPs were identified as Lin⁻c-Kit⁺CD16/CD32^{lo}CD34^{lo}CD41⁻, and MkPs were identified as Lin⁻c-Kit⁺CD16/CD32^{lo}CD34^{lo}CD41⁺.

Expression of c-MPL on late-stage megakaryocytes was detected by staining bone marrow cells with a cocktail of lineage markers (Sca1, CD127, CD45R, CD19, CD3e, TER-119, CD2, CD8 and Ly6-C/G, all FITC conjugated; eBioscience), CD41 (eBioscience) and c-MPL or isotype control as above. After staining with the antibodies, the bone marrow cells were then fixed and permeabilized using BD cytofix/perm buffer for 20 min on ice followed by washing with BD cytofix/perm wash buffer. Cells were then resuspended in FACS buffer containing propidium iodide to determine megakaryocyte ploidy. Expression of c-MPL was measured on total and late-stage megakaryocytes (defined as 32N and 64N).

Expression of c-MPL on platelets was assessed by obtaining platelet-rich plasma and staining with CD41 and c-MPL as outlined above. The surface expression of c-MPL on platelets was then quantified by MFI normalized to the isotype control.

c-Cbl Phosphorylation.

Bone marrow progenitor cells were stimulated with TPO at the indicated concentration for the specified period of time at 37° C. and then immediately diluted with ice-cold buffer and placed on ice to prevent further changes in phosphorylation. Cells were then centrifuged at 800 g for 2 min, and the pellet was resuspended in BD fix buffer (BD Biosciences) for 10 min on ice. The cells were washed with BD flow cytometry staining buffer, centrifuged, and then resuspended in BD cytofix/perm buffer III for 20 min. After this, the cells were washed and resuspended in BD staining buffer and incubated with lineage (Sca1, CD127, CD45R, CD19, CD11b, CD3e, TER-119, CD2, CD8, CD4 and Ly-6G, all FITC; eBioscience) and progenitor cell markers (c-Kit, CD16/CD32 (FcγRII/III), CD34, CD41 and antibody to p-c-CBL (Tyr700 human, Tyr698 mouse; BD Biosciences)) or an isotype control for 30 min on ice. The cells were then washed, resuspended in FACS buffer and run on an LSRII. The amount of phosphorylated c-Cbl was normalized against that of isotype control staining.

Immunofluorescence Confocal Microscopy.

MkPs collected by FACS from WT or Abcg4−/− bone marrow cells were attached to glass slides by a brief spin in Cytospin. The cells were then fixed with 2% paraformaldehyde, permeabilized with 1% Triton X-100 in PBS for 1 min and incubated with 4% BSA in PBS plus 0.1% saponin to block nonspecific binding sites. Diluted primary antibodies against ABCG4 or cellular organelle markers (58K Golgi protein-specific antibody, Novus Biologicals; TGN38-specific antibody, BD Biosciences; c-MPL-specific antibody, Sigma-Aldrich; Lamp2-specific antibody, Novus Biologicals) were then added to the cells in 1:200 dilution and incubated at room temperature for 2 h. After washing, fluorescent secondary antibodies (1:400 dilution) were added and incubated for 1 h. Where indicated, the washed cells were counterstained with or without DAPI and examined with a fluorescence confocal microscope.

Statistics.

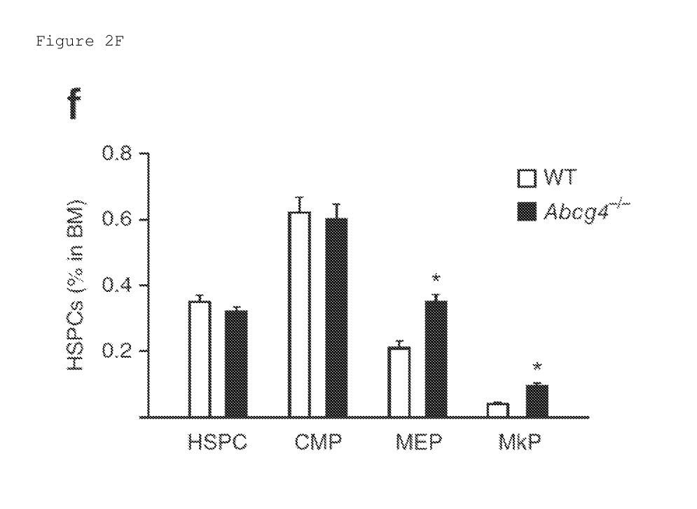

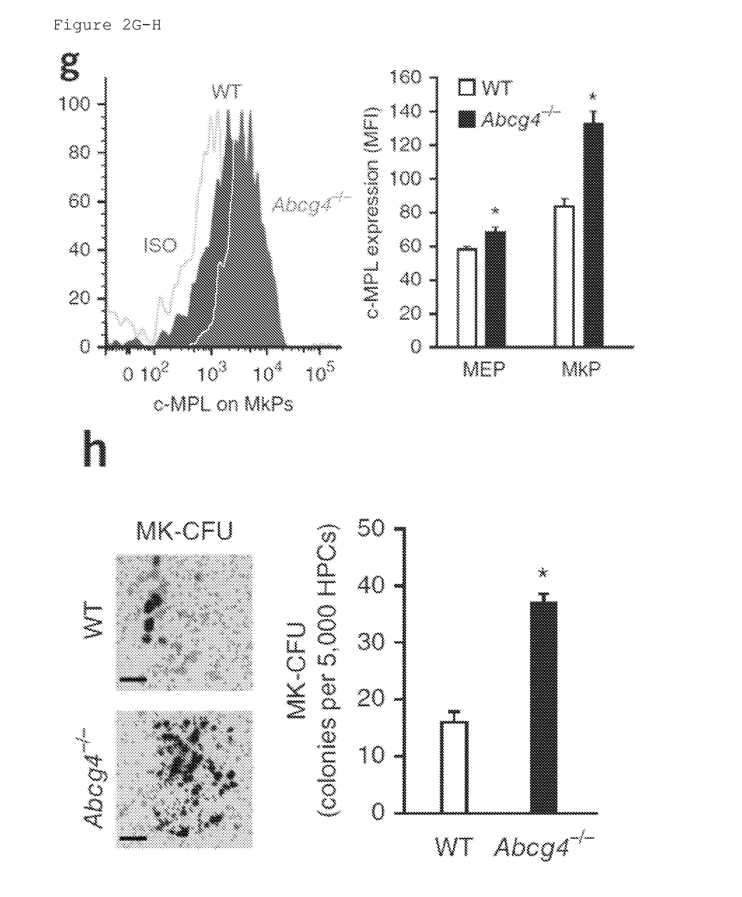

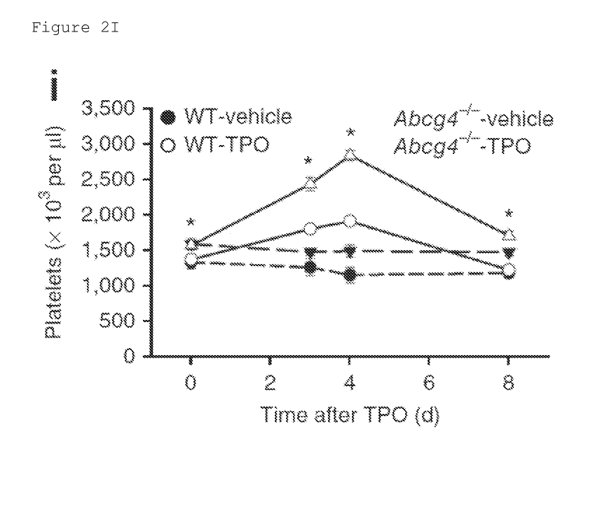

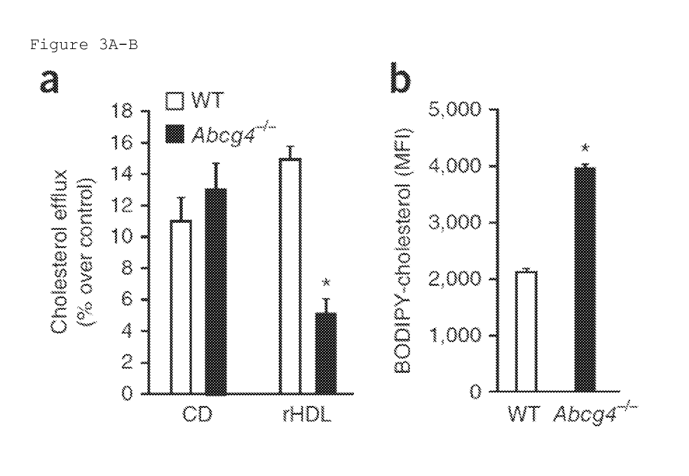

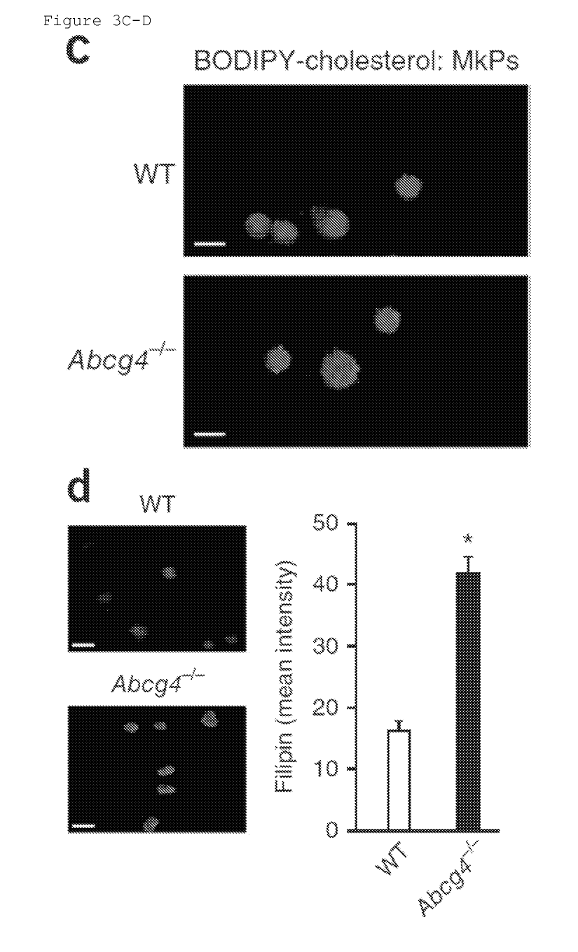

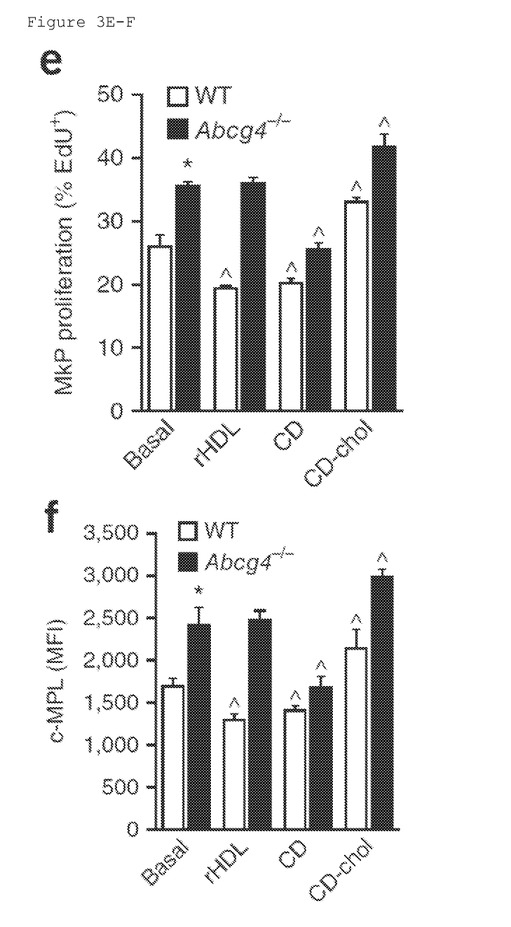

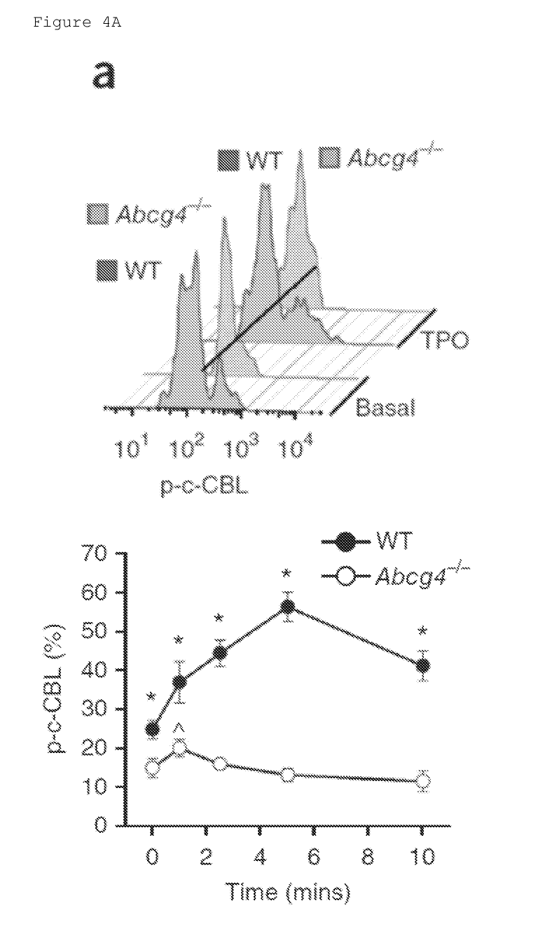

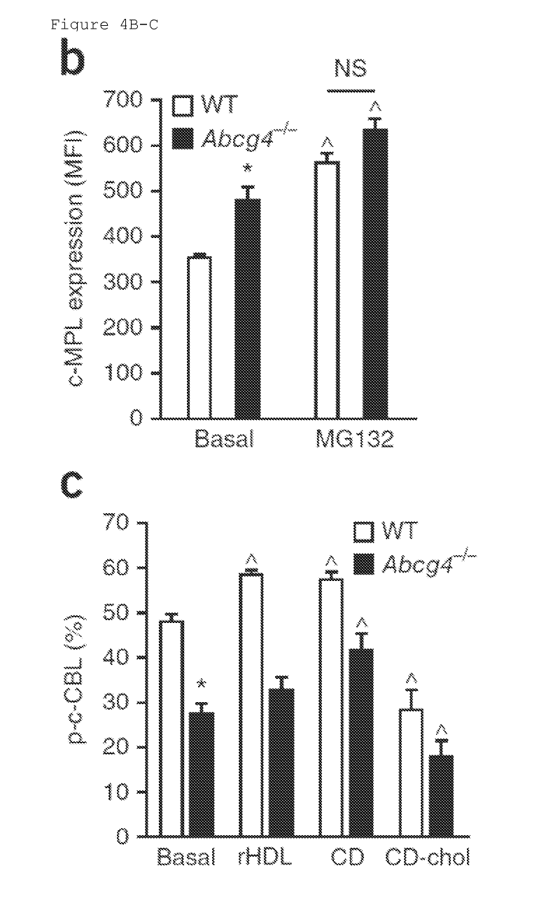

For aortic morphometric atherosclerotic lesion quantification and analysis, two-way analysis of variance (ANOVA) was used. For comparison of one group with another, for instance in the c-CBL phosphorylation time course experiment (FIG. 4a), a t test was used. For comparison of various treatments on different genotypes, one-way ANOVA was used

### Example 1

### ABCG4 Deficiency Accelerated Atherosclerosis and Thrombosis

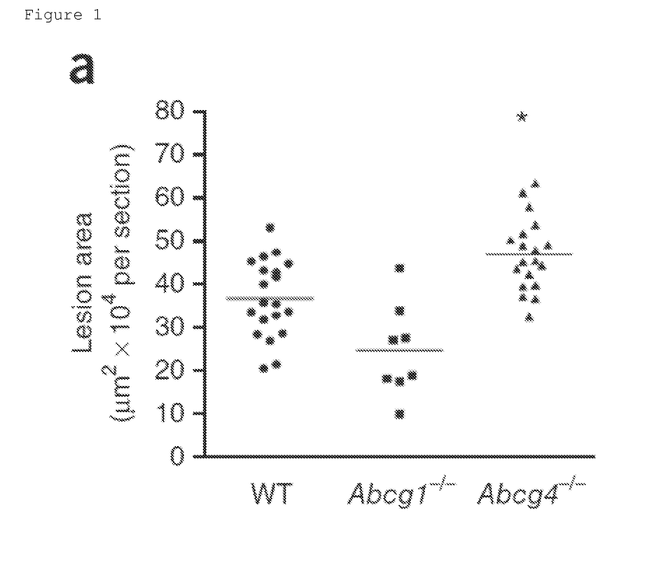

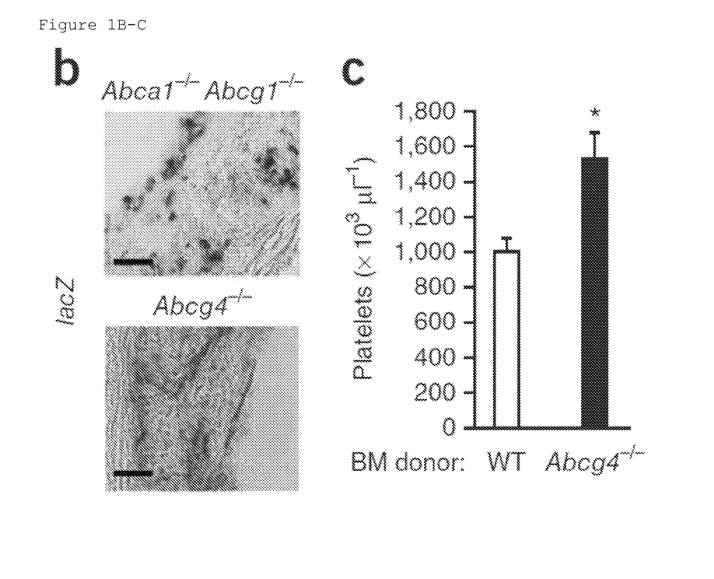

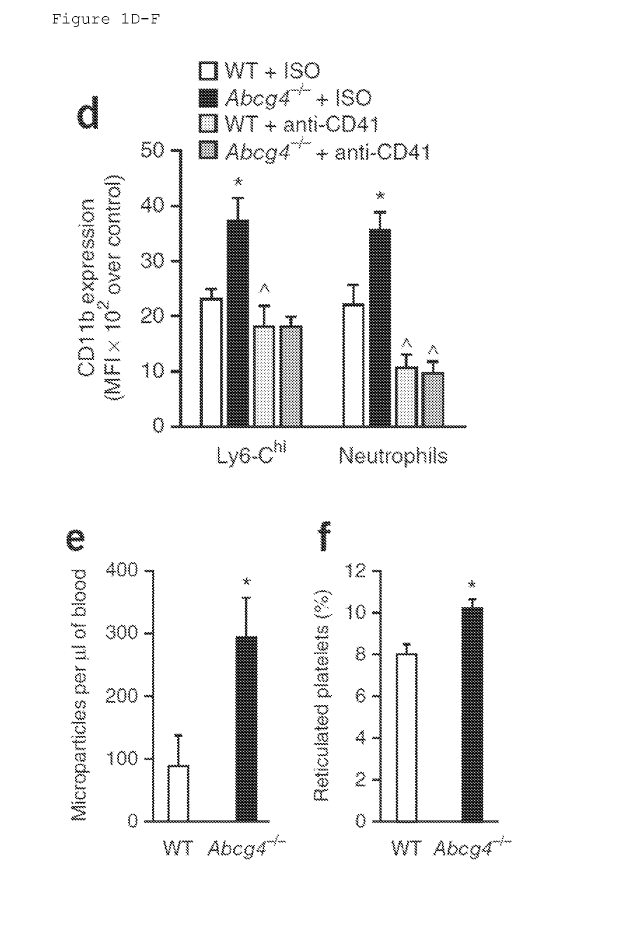

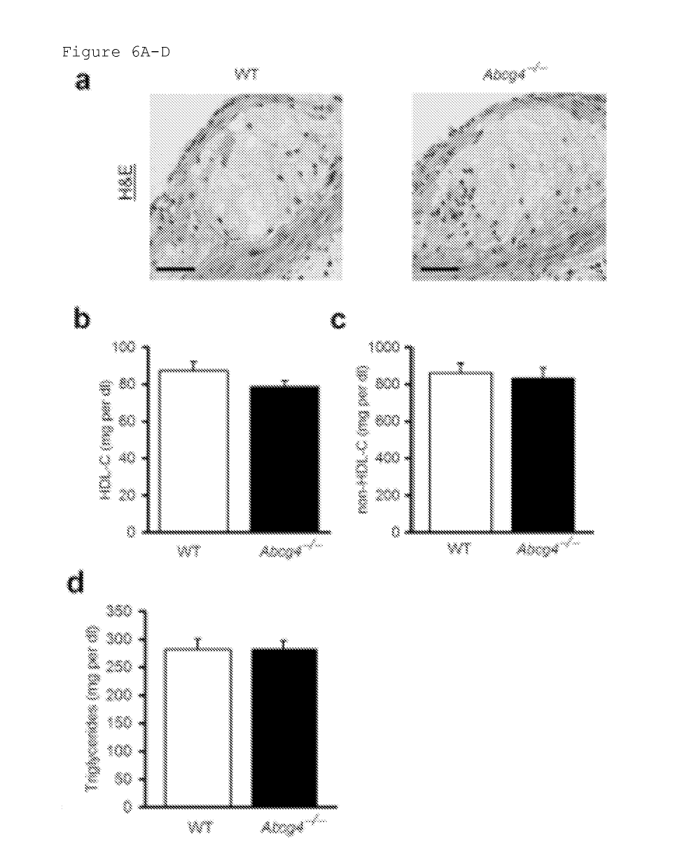

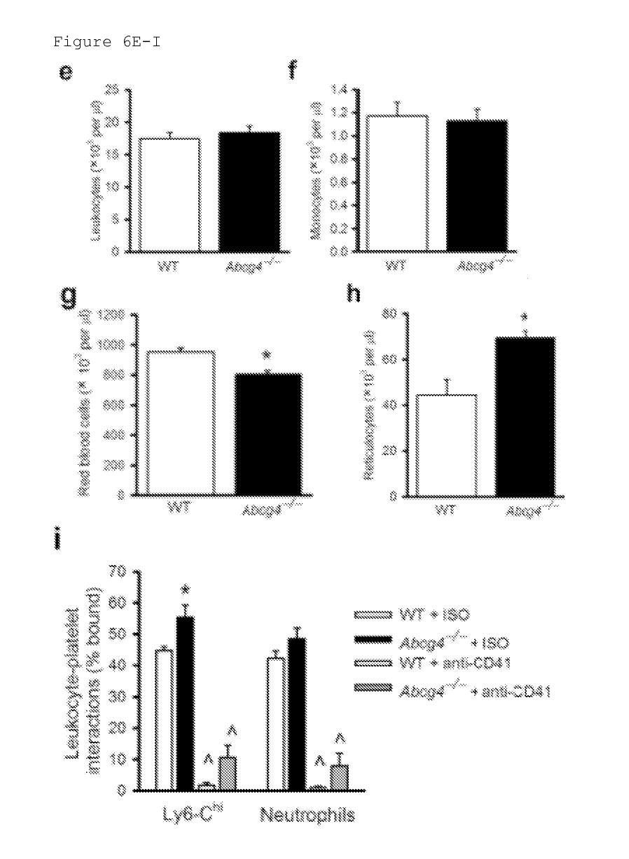

Hematopoietic parameters and atherogenesis in a hypercholesterolemic mouse model of atherosclerosis were assessed by reconstituting irradiated Ldlr−/− mice with bone marrow from wild-type (WT) or Abcg4−/− mice. Atherosclerosis studies were performed in Ldlr−/− mice transplanted with Abcg1−/− bone marrow. After the mice had been fed a high-fat, high-cholesterol diet (WTD) for 12 weeks, atherosclerotic lesion size was significantly increased in the aorta of Ldlr−/− mice receiving bone marrow transplantation (BMT) with ABCG4-deficient bone marrow (FIG. 1a). In contrast, mice receiving ABCG1-deficient bone marrow did not show increased advanced atherosclerosis (FIG. 1a), consistent with previous studies (Ranalletta, M. et al. 2006; Meurs, I. et al. 2012). Histological analysis of the lesions showed typical, macrophage foam cell-rich atherosclerotic lesions with no differences in morphology between the BMT groups (FIG. 6a). The Abcg4 knockout mice used were generated using a lacZ knock-in allele at the Abcg4 locus. However, no lacZ-positive cells in lesions of mice receiving Abcg4−/− bone marrow were found (FIG. 1b). As a positive control, aortic lesions from Ldlr−/− mice receiving Abca1−/− Abcg1−/− bone marrow (also generated with a lacZ knock-in allele in the Abcg1 locus) (Yvan-Vharvet, L. et al. 2010) were stained and lacZ-positive cells were found indicating Abcg1 expression in the lesions. Plasma lipid and lipoprotein concentrations were similar in recipients of WT or Abcg4−/− bone marrow (FIG. 6b-d), as were leukocyte, monocyte (FIG. 6e,f), total lymphocyte, B cell and T cell counts (data not shown). Platelet counts were 52% greater in Abcg4−/− bone marrow recipients compared with recipients of WT bone marrow (FIG. 1c). Mild anemia and reticulocytosis was observed in the Ldlr−/− mice receiving Abcg4−/− bone marrow (FIG. 1g,h).

Activated platelets contribute directly to atherogenesis (Huo, Y. et al. 2003), in part by promoting activation and adhesion of monocytes to the arterial endothelium (Koenen, R. R. et al. 2009; Huo, Y. et al. 2003). The numbers of platelet-neutrophil and platelet-Ly6-C^{hi} monocyte aggregates were increased in hypercholesterolemic mice receiving Abcg4−/− bone marrow compared to those receiving WT bone marrow (FIG. 6i). These aggregated leukocytes from the recipients of Abcg4−/− bone marrow expressed higher levels of CD11b (as determined by mean fluorescence intensity (MFI)), a key cell adhesion molecule that facilitates adhesion to the endothelium20 (FIG. 1d), indicating they were more activated than in the WT bone marrow recipients. Depletion of platelets by injection of CD41-specific antibodies, which markedly reduced platelet count in Ldlr−/− mice receiving WT or Abcg4−/− bone marrow (data not shown), reduced aggregate numbers and leukocyte CD11b expression (FIG. 1d and FIG. 6i). Platelet microparticles promote atherogenesis by facilitating chemokine deposition onto the arterial endothelium and recruiting monocytes to lesions (Mause, S. F. et al. 2005). The numbers of platelet-derived microparticles were threefold higher in hypercholesterolemic mice receiving Abcg4−/− bone marrow than in those receiving WT bone marrow (FIG. 1e). Circulating amounts of reticulated platelets correlate directly with platelet reactivity (Guthikonda, S. et al. 2008) and are strongly associated with increased risk of myocardial infarction in humans (Lakkis, N. et al. 2004). There was also a significant increase in the percentage of reticulated platelets in mice receiving Abcg4−/− bone marrow (FIG. 1f), consistent with increased platelet production and turnover (Stohlawetz, P. et al. 1998). These findings are consistent with previous studies in which infusions of activated platelets increased atherosclerotic lesion formation (Huo, Y. et al. 3003) and suggest that increased endogenous platelet production in recipients of Abcg4−/− bone marrow leads to accelerated atherogenesis.

Thrombocytosis and increased amounts of reticulated platelets would also be expected to promote thrombosis. Mice are resistant to spontaneous thrombosis on atherosclerotic plaques. Thus, to assess thrombogenicity, thrombus formation was evaluated in whole blood using an ex vivo perfusion chamber model. Compared to WT controls, a marked increase in Abcg4−/− platelet adhesion and aggregation to a collagen-coated surface under shear-flow conditions was found (FIG. 1g). Arterial thrombosis in vivo was examined using a carotid artery thrombosis model. Carotid artery occlusion by a thrombus after injury with FeCl₃ was significantly accelerated in mice receiving Abcg4−/− bone marrow compared to those receiving WT bone marrow (FIG. 1h). Together these findings indicate an increased propensity to thrombus formation in hypercholesterolemic mice with bone marrow ABCG4 deficiency.

### Example 2

### ABCG4 is Expressed in Platelet Progenitors

It was considered that ABCG4 might be acting in platelets to influence cholesterol efflux and platelet numbers. However, Abcg4 mRNA was not detected in WT platelets or lacZ staining in platelets of Abcg4−/− mice. In Abcg4−/− mice, there was no alteration in cholesterol efflux by platelets to HDL or in platelet cholesterol concentrations (FIG. 7a,b), indicating that ABCG4 does not act in platelets to regulate circulating platelet numbers. Thus, the mechanisms by which ABCG4 acts to regulate platelet numbers seem to be distinct from those reported for the scavenger receptor SR-BI (Nofer, J. R. et al. 2001).

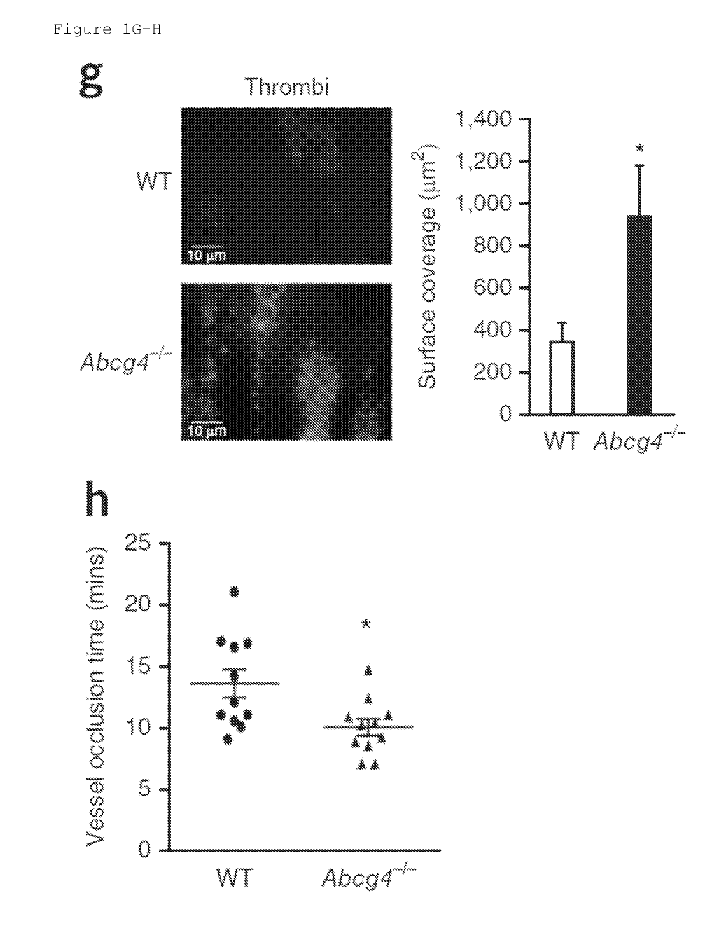

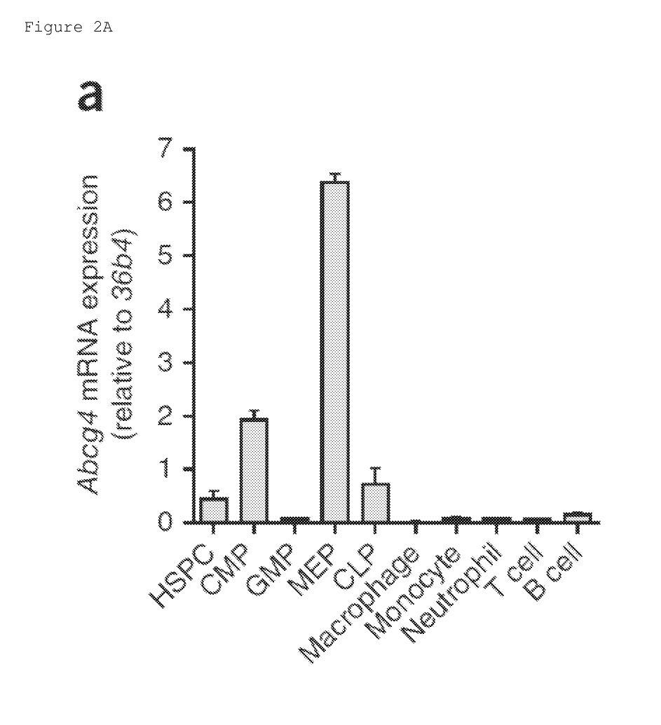

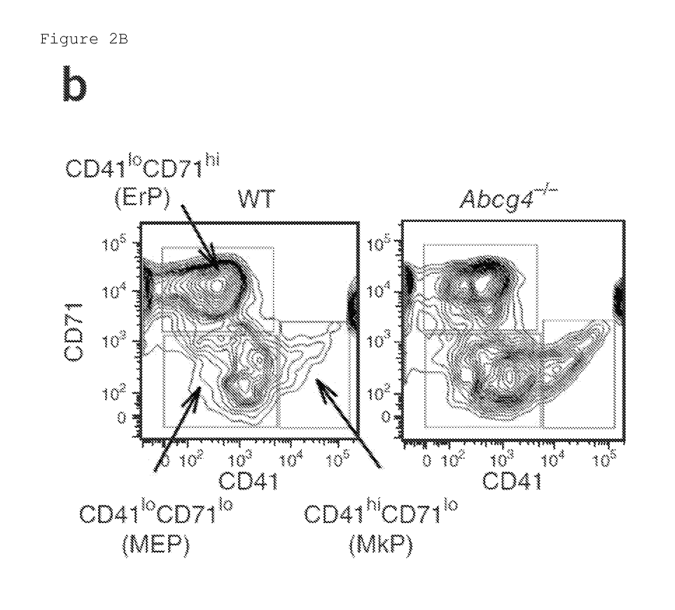

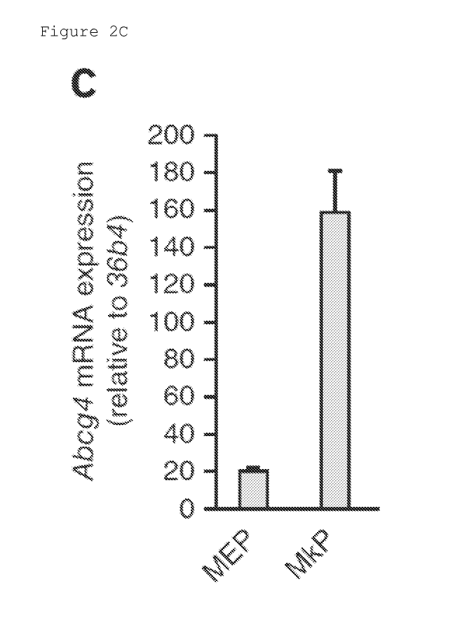

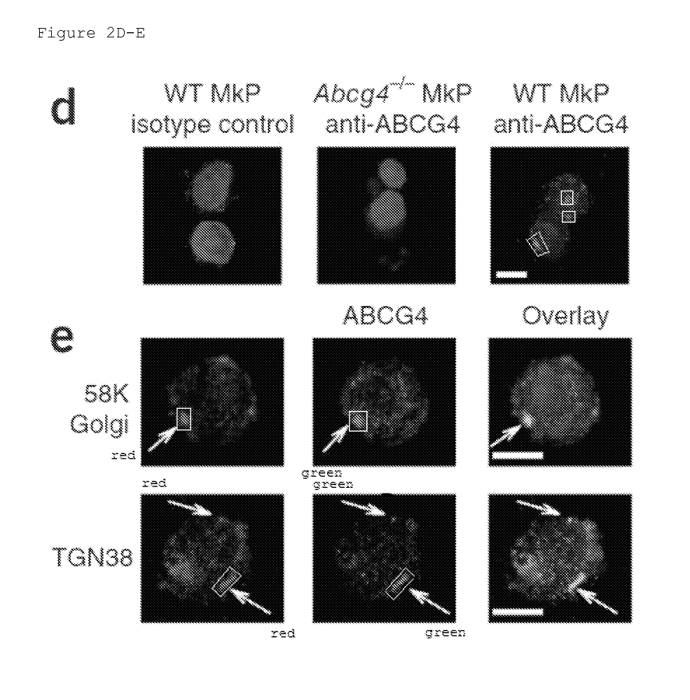

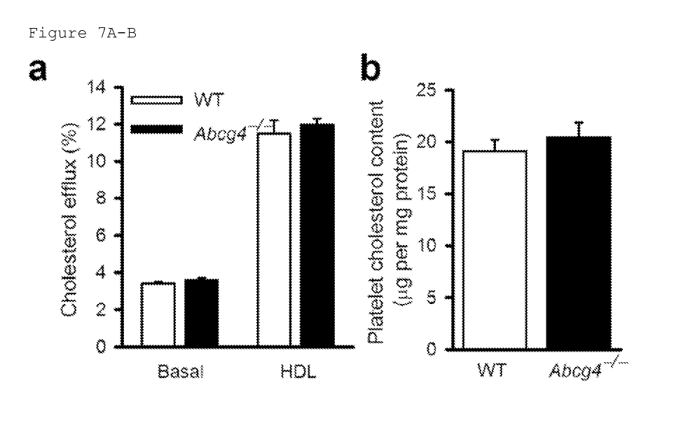

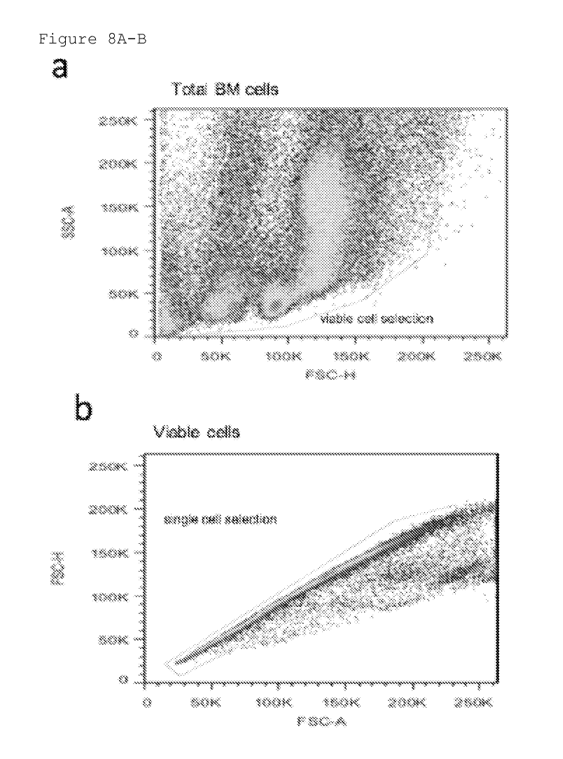

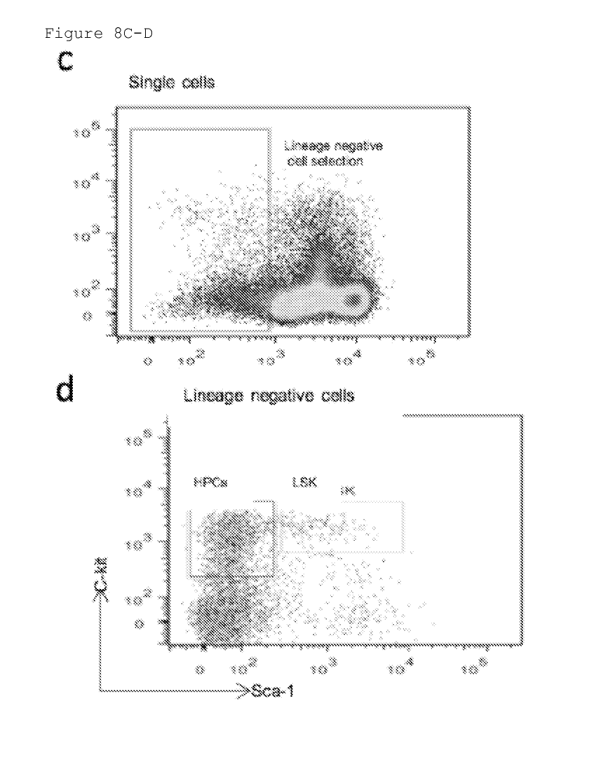

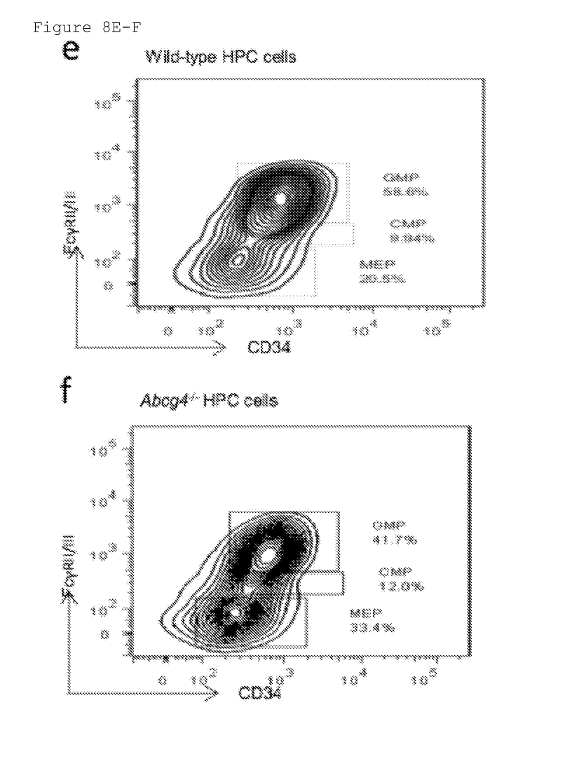

The phenotype of ABCG4-deficient mice, including prominent thrombocytosis, mild anemia and increased numbers of reticulated platelets, platelet and leukocyte aggregates and platelet microparticles, resembles that of essential thrombocytosis (Villmow, T. et al. 2002), a myeloproliferative neoplasm in which mutations in the genes encoding c-MPL or JAK2 in bone marrow progenitors lead to excessive proliferation of platelet progenitors and increased platelet production (Tefferi, A. et al. 2011; Pikman, Y. et al. 2006). Platelets are produced by megakaryocytes in the bone marrow and spleen, and megakaryocytes are derived from megarkaryocyte-erythrocyte progenitors (MEPs). It was hypothesized that ABCG4 might be expressed in bone marrow platelet progenitors and could be involved in the regulation of their proliferation and in megakaryocytopoiesis. After separation of bone marrow hematopoietic cell populations by FACS (FIG. 8), Abcg4 mRNA were detected primarily in MEPs (FIG. 2a), with a lower level of expression in the common myeloid progenitor (CMP) population. Very low or no Abcg4 expression was found in other cell types (FIG. 2a). The restricted expression of Abcg4 to MEPs contrasts with the expression of Abca1 and Abcg1, which are highly expressed in hematopoietic stem and progenitor cells (HSPCs) but not MEPs. To test whether Abcg4 is the main cholesterol efflux transporter expressed in MEPs, mice were treated with a liver X receptor (LXR) activator in the attempt to induce Abca1 and Abcg1 expression; however, there was little expression of these two genes in, MEPs of the treated mice, suggesting that Abcg4 is the dominant transporter in these cells (FIG. 9a,b).

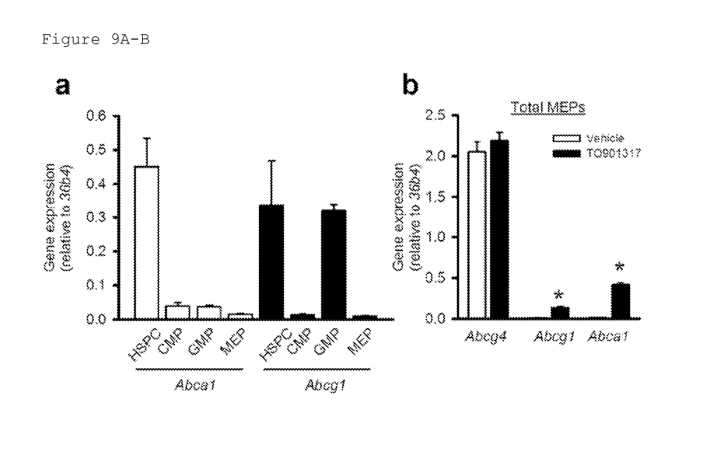

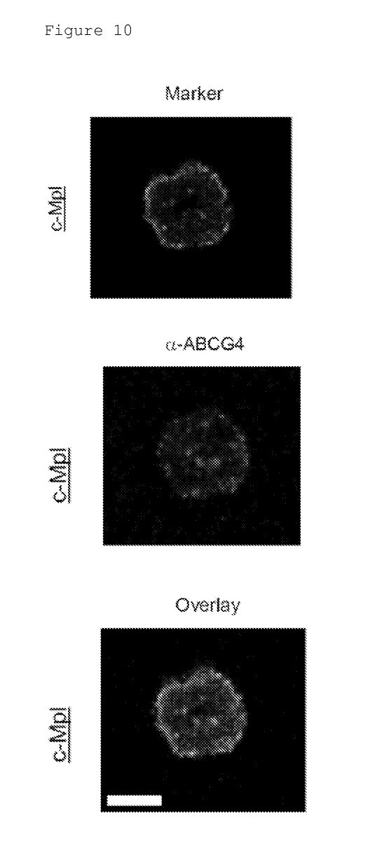

Recent studies have shown that the MEP population contains CD41⁺ cells with megakaryocyte progenitor potential, as well as CD71+ cells with erythrocyte progenitor potential (Frontelo, P. et al. 2007). The MEP population were further sorted into CD41⁺CD71^{lo}, CD41^{lo}CD71⁺ and CD41^{lo}CD71^{lo} cell populations (FIG. 2b) and CD41⁺CD71^{lo} cells are referred to as MkPs. High Abcg4 expression in MkPs (FIG. 2c) and CD41^{lo}CD71^{lo} cells and lower expression in CD41^{lo}CD71^{lo} cells was found (FIG. 2c). Immunofluorescence confocal microscopy was used to assess ABCG4 protein expression and localization in the MkPs. Specific ABCG4 staining was detected in WT MkPs with ABCG4-specific antibody but not in Abcg4−/− MkPs or WT MkPs stained with isotype-matched control antibody (FIG. 2d). Notably, ABCG4 staining partially colocalized with Golgi and, particularly, trans-Golgi markers (FIG. 2e), whereas no colocalization with c-MPL (plasma membrane), Lamp2 (lysosome) or calnexin (endoplasmic reticulum) was detected (FIG. 10). Thus, ABCG4 is selectively expressed in the MEP and MkP populations and seems to localize partly to the trans Golgi.

### Example 3

### ABCG4 Deficiency Increased MkP Proliferation and Promotes Megakaryopoiesis

The percentages of MkPs and CD41loCD71lo MEPs, but not of HSPCs or CMPs, were significantly increased in the bone marrow of hypercholesterolemic recipients of Abcg4−/− bone marrow compared to recipients of WT bone marrow (FIG. 2f). The numbers of CD41^{lo}CD71⁺ erythrocyte progenitors were also increased in these mice (data not shown). mRNA levels of Gatal, PU.1 (also known as Sfpi1), Eklf (also known as Klf1) and Fli1, transcription factors known Co have crucial roles in the regulation of MEP, MkP and erythrocyte progenitor cell proliferation and differentiation, were similar in Abcg4−/− CD41^{lo}CD71^{lo} MEPs, CD41⁺CD71^{lo} MkPs and CD41^{lo}CD71⁺ erythrocyte progenitors, suggesting that the lineage choice of Abcg4−/− hematopoietic cells is not markedly altered.

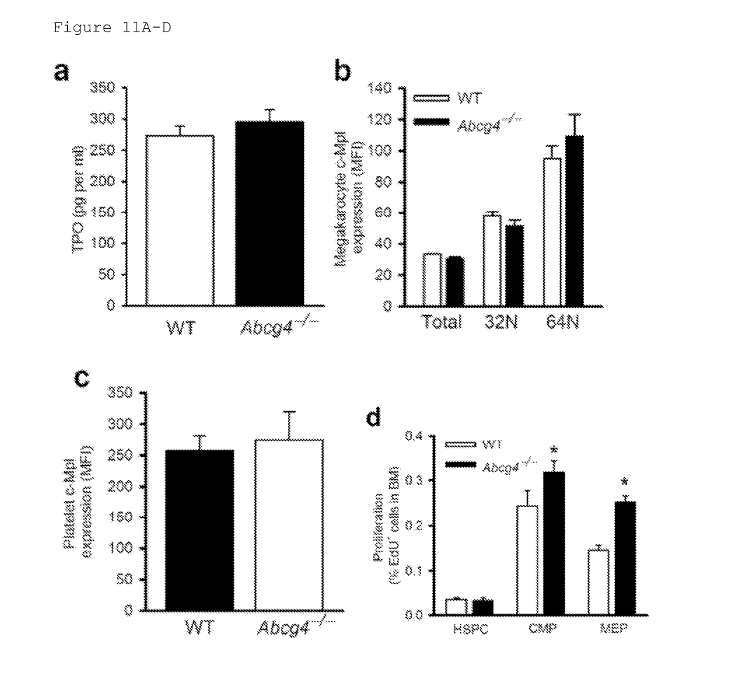

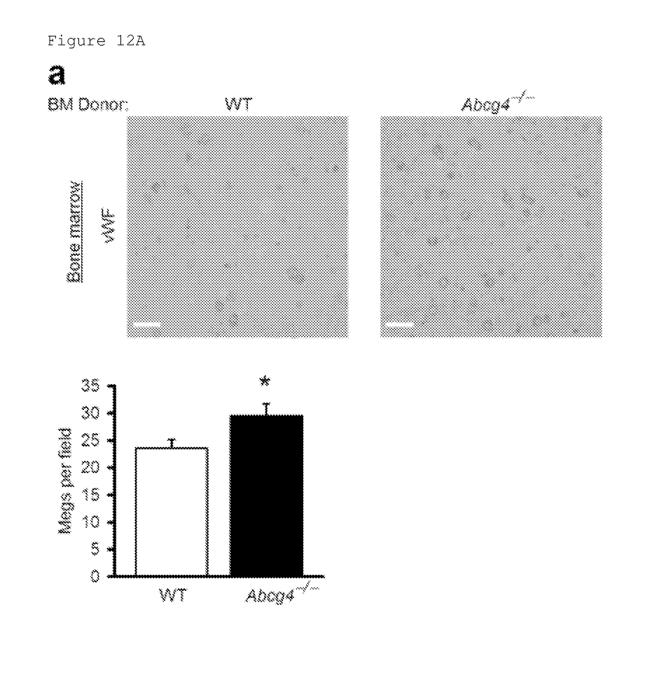

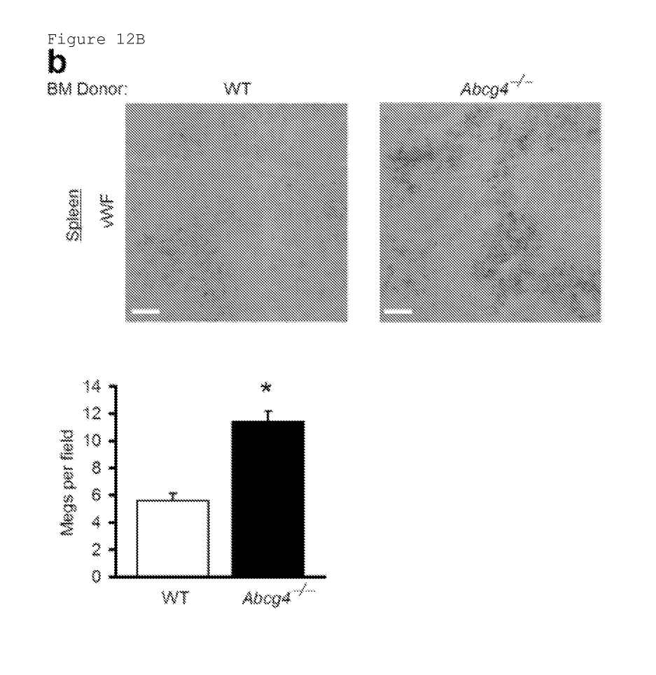

TPO is the most important growth factor regulating megakaryocyte and platelet lineage development in vivo. We did not observe any change in plasma TPO concentrations in mice receiving Abcg4−/− bone marrow compared to those receiving WT bone marrow (FIG. 11a). However, increased expression of c-MPL on the surface of Abcg4−/− MkPs and CD41loCD71lo MEPs (FIG. 2g) was found but not on megakaryocytes or platelets (FIG. 11b,c). This is consistent with the expression profile of Abcg4 and the hypothesis that increased MkP proliferation is the underlying mechanism of thrombocytosis in ABCG4-deficient mice. Indeed, increased EdU incorporation into DNA was increased in MEPs from Abcg4−/− mice compared to those from WT mice (FIG. 11d). Colony-formation assays showed a 2.5-fold increase in the number of megakaryocyte colonies arising from ABCG4-deficient compared to WT bone marrow in response to TPO (FIG. 2h). Moreover, the number of megakaryocytes was increased in the bone marrow and spleen of Ldlr−/− mice receiving Abcg4−/− bone marrow compared to those receiving WT bone marrow (FIG. 12a,b).

### Example 4

### Increased Thrombopoietin-Induced Platelet Production in Abcg4−/− Mice

Platelet counts are tightly regulated by a negative feedback mechanism in which c-MPL at the surface of megakaryocytes and platelets serves as a clearance sink for TPO and thus limits the increase in platelet count that results from increased TPO-c-MPL signaling in bone marrow cells (Hitchock, I. S. et al. 2008; Tiedt, R. et al. 2009). TPO administration to mice may overwhelm the negative feedback regulatory mechanism, uncovering the effects of increased c-MPL activity (Kelemen, E. et al. 1999). To test the hypothesis that ABCG4 deficiency in MEPs and MkPs results in increased cell surface expression of c-MPL, increased sensitivity of cells to TPO and enhanced platelet production, TPO was administered to WT and Abcg4−/− mice. The increase in the number of platelets was much more pronounced in Abcg4−/− mice (2.1-fold) compared to WT mice (1.4-fold) (FIG. 2i). These results indicate that ABCG4 deficiency renders mice more responsive to TPO in vivo, consistent with the idea that increased c-MPL expression on MkPs is the mechanism underlying increased platelet production in Abcg4−/− mice.

### Example 5

### ABCG4 Promoted Cholesterol Efflux from MkPs to HDL

Potential mechanisms linking ABCG4 deficiency to increased expression of c-MPL and increased proliferation and expansion of MkPs was investigated. Cellular cholesterol efflux from WT and Abcg4−/− MkPs was examined using a fluorescent cholesterol analog (BODIPY-cholesterol)-based flow cytometry assay. ABCG4 deficiency was associated with reduced cholesterol efflux to reconstituted HDL (rHDL) in Abcg4−/− MkPs (FIG. 3a). BODIPY-cholesterol concentrations in Abcg4−/− MkPs were also significantly increased (FIG. 3b). These findings could indicate that ABCG4-deficiency resulted in defective cholesterol efflux or that membranes of Abcg4−/− cells have a higher affinity, capacity or both for cholesterol. A significant portion of the BODIPY-cholesterol that accumulated in Abcg4−/− MkPs was in the plasma membrane (FIG. 3c). Free cholesterol content as assessed by filipin staining was also substantially increased in the plasma membrane of Abcg4−/− MkPs (FIG. 3d). Cholesterol accumulation is known to suppress the expression of cholesterol-responsive genes (Brown, M. S. et al. 2009). Accordingly, expression of the cholesterol-responsive genes Ldlr and Hmgcs1 was significantly (P<0.05) decreased in Abcg4−/− relative to the WT MEPs; however, this effect was not observed in granulocyte-macrophage progenitors, which do not express Abcg4 (FIG. 13a,b). Thus, despite findings showing that ABCG4 localizes to the Golgi (FIG. 2e), ABCG4 deficiency results in defective cholesterol efflux to HDL and an increase in cell cholesterol content, including in the plasma membrane. These results are consistent with studies suggesting that sterol-rich plasma membrane lipid raft domains can be segregated from non-raft domains in the trans Golgi (Lingwood, et al. 2010).

To determine whether an increase in cellular cholesterol content can recapitulate the effects of ABCG4 deficiency, cells were loaded with cholesterol-cyclodextrin complexes. This led to increased proliferation of WT and Abcg4−/− MkPs, paralleling increased cell surface c-MPL expression (FIG. 3e,f). After treatment of the cells with cyclodextrin to remove cellular cholesterol, proliferation and the cell surface expression of c-MPL in Abcg4−/− MkPs were significantly reduced to levels similar to those in cyclodextrin-treated WT MkPs (FIG. 3e,f). Although rHDL significantly reduced WT MkP proliferation and cell surface expression of c-MPL, it had no effect in Abcg4−/− MkPs, consistent with the cholesterol efflux data (FIG. 3a). In addition, removal of cellular cholesterol by cyclodextrin reversed the increase in the number of magakaryecyte colonies associated with ABCG4 deficiency (FIG. 13c). These findings suggest that ABCG4 acts to modulate MkP cell surface c-MPL expression and cell proliferation by regulation of membrane cholesterol content.

### Example 6

### Decreased Downregulation of TPO Receptor in Abcg4−/− MkPs

Mechanisms linking changes in cellular cholesterol concentrations to altered c-MPL expression in MkPs were also studied. Previous studies have shown that TPO binding to its receptor, c-MPL, results in activation of a negative feedback loop in which c-CBL-mediated ubiq-uitinylation leads to receptor internalization, degradation or both (Saur, S. J. et al. 2010). c-CBL phosphorylation in response to the activation of growth factor receptors is required to mediate negative feedback regulation (Nadeau, S. et al. 2012). It was assessed whether such negative feedback regulation is defective in Abcg4−/− MkPs. In response to TPO treatment, the increase in the amount of c-CBL tyrosine phosphorylation was markedly blunted in Abcg4−/− compared to WT MkPs (FIG. 4a), whereas the amount of total c-CBL was unchanged. Treatment of WT MkPs with a proteasome inhibitor, MG132, increased c-MPL expression to a level similar to that in Abcg4−/− cells (FIG. 4b), consistent with the idea that in Abcg4−/− there is decreased proteasomal degradation of c-MPL compared to WT cells. Cholesterol loading by cholesterol-cyclodextrin complexes reduced c-CBL phosphorylation, whereas removal of cellular cholesterol by cyclodextrin increased c-CBL phosphorylation in both WT and Abcg4−/− MkPs (FIG. 4c). Although rHDL treatment increased the amount of phosphorylated c-CBL in WT MkPs, it did not alter c-CBL phosphorylation in Abcg4−/− MkPs (FIG. 4c), consistent with the inability of rHDL to modulate c-MPL expression and cell proliferation of Abcg4−/− MkPs. These findings suggest that impaired cholesterol efflux in Abcg4−/− MkPs results in defective c-CBL-mediated feedback downregulation of c-MPL by TPO.

### Example 7

### LYN Kinase Modulated Proliferative Responses of MkPs

The kinase(s) catalyzing c-CBL tyrosine phosphorylation in response to TPO are not known, SRC-family kinases (SFKs) such as LYN, FYN and c-SRC are known to phosphorylate tyrosine residues of c-CBL36, leading to its activation, and SFR inhibitors have been shown to increase cell surface c-MPL expression through undefined mechanisms (Hitchcock, I. S. et al. 2008). It was hypothesized that the activity of SFKs is decreased in Abcg4−/− MkPs, leading to decreased c-CBL phosphorylation. Consistent with this suggestion, treatment of WT and Abcg4−/− MkPs with SU6656, an inhibitor of LYN, FYN and c-SRC37, markedly decreased c-CBL phosphorylation, increased cell surface expression of c-MPL and abolished the difference in response to TPO between WT and Abcg4−/− MkPs (FIG. 4d,e). TPO activation of c-MPL increases the kinase activity of LYN and FYN but not other SFKs (Lannutti, B. J. et al. 2003). LYN kinase is palmitoylated and membrane associated, and its activity is increased by decreased membrane cholesterol content (Oneyama, C. et al. 2009). Notably, Lyn−/− mice show increased megakaryocytopoiesis with mild thrombocytosis (Lannutti, B. J. et al. 2006) and mild anemia with reticulocytosis (Ingley, E. et al. 2005), defects that bear a striking resemblance to those of Abcg4−/− mice. Thus it was hypothesized that LYN might be the dominant tyrosine kinase catalyzing c-CBL tyrosine phosphorylation in response to TPO. TPO-treated Lyn−/− MkPs showed decreased c-CBL phosphorylation and increased cell surface c-MPL expression (FIG. 4f,g) and cell proliferation compared to WT MkPs (FIG. 4h), demonstrating that LYN has a key role in regulating the tyrosine phosphorylation of c-CBL and in MkP proliferation in response to TPO. Cholesterol loading by cholesterol-cyclodextrin complexes decreased c-CBL phosphorylation, increased c-MPL expression and enhanced cell proliferation in WT MkPs but had no effect in Lyn−/− MkPs (FIG. 4f-h). Treatments with either cyclodextrin or rHDL to induce cholesterol efflux decreased the proliferation of WT MkPs. In contrast, Lyn−/− MkPs showed increased proliferation that was unresponsive to either cholesterol loading or depletion treatments (FIG. 4h). These findings indicate an essential role of LYN kinase in mediating the effects of cholesterol loading and unloading on c-CBL phosphorylation as well as the effects of TPO on c-MPL expression and MkP proliferation.

### Example 8

### A LYN Kinase Activator Reduced c-MPL Expression

To further assess the possible involvement of LYN in the negative regulation of surface c-MPL expression on MkPs, the effects of pharmacological LYN activation were tested. Treatment of bone marrow cells from hypercholesterolemic Ldlr−/− recipient mice with tolimidone, a compound that selectively increases LYN kinase activity in vivo (Saporito, M. S. et al. 2012), reduced cell surface c-MPL expression in both WT and Abcg4−/− MkPs (FIG. 4i) and completely reversed the increased cell surface expression of c-MPL in Abcg4−/− MkPs from normocholesterolemic mice (FIG. 14). Together these observations suggest that in the presence of excessive membrane cholesterol accumulation, decreased LYN kinase activity leads to diminished c-CBL-mediated downregulation of c-MPL by TPO.

known TPO-mediated signaling pathways that could potentially be activated in Abcg4−/− MkPs were assessed. Both basal and TPO-stimulated amounts of phosphorylated ERK1/2 and phosphorylated Akt were significantly higher in Abcg4−/− compared to WT MkPs; the amounts of phosphorylated STAT5 were also increased, albeit nonsignificantly (FIG. 4j). This pattern is similar to that seen with LYN deficiency (Lannutti, B. J. et al. 2006).

### Example 9

### HDL Infusion Decreased MkP Proliferation and Platelet Count

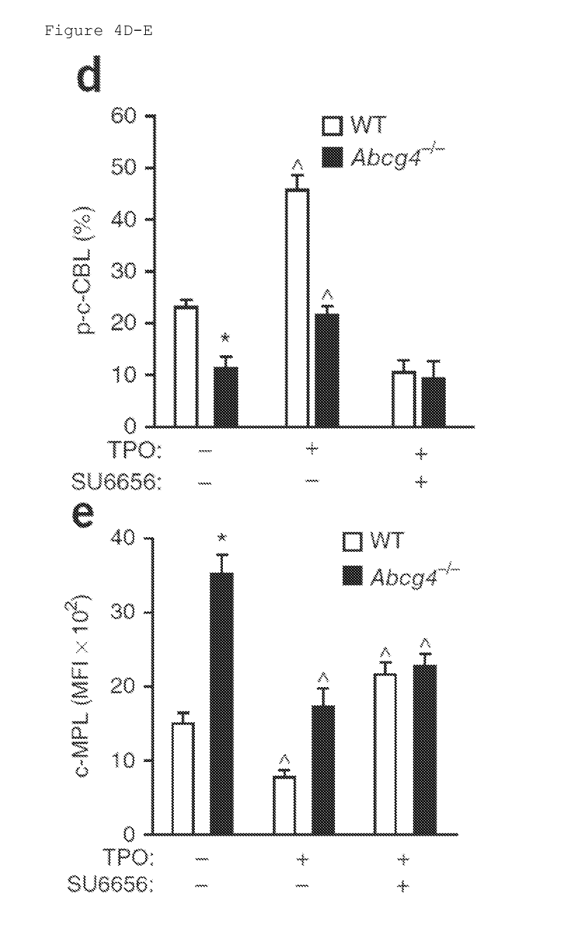

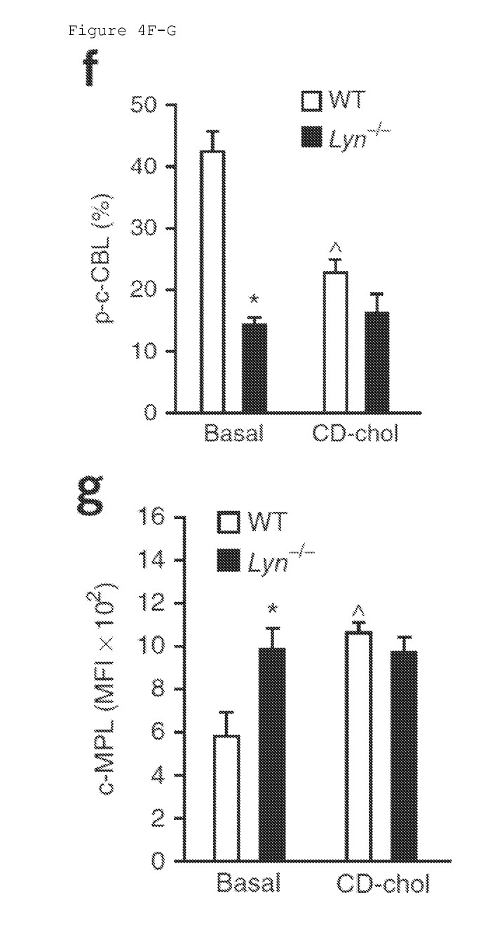

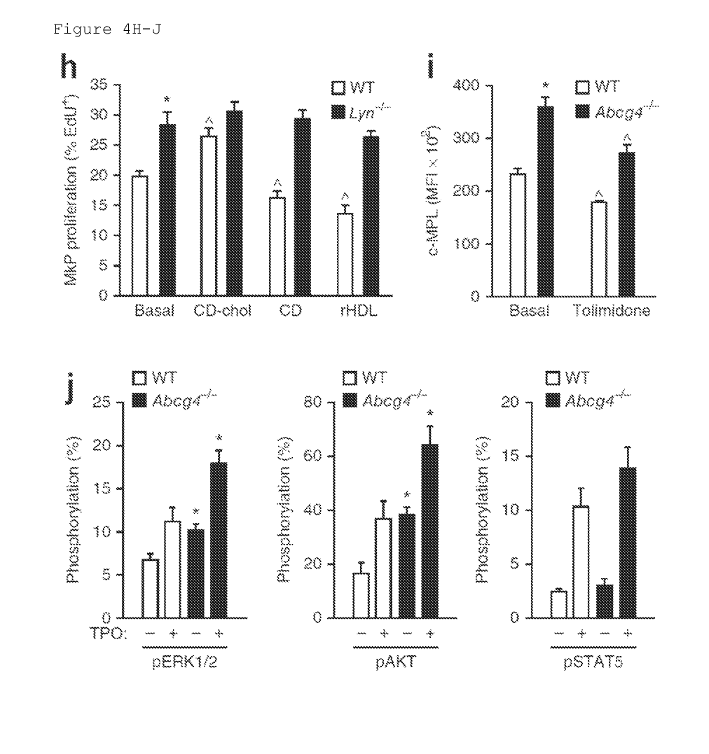

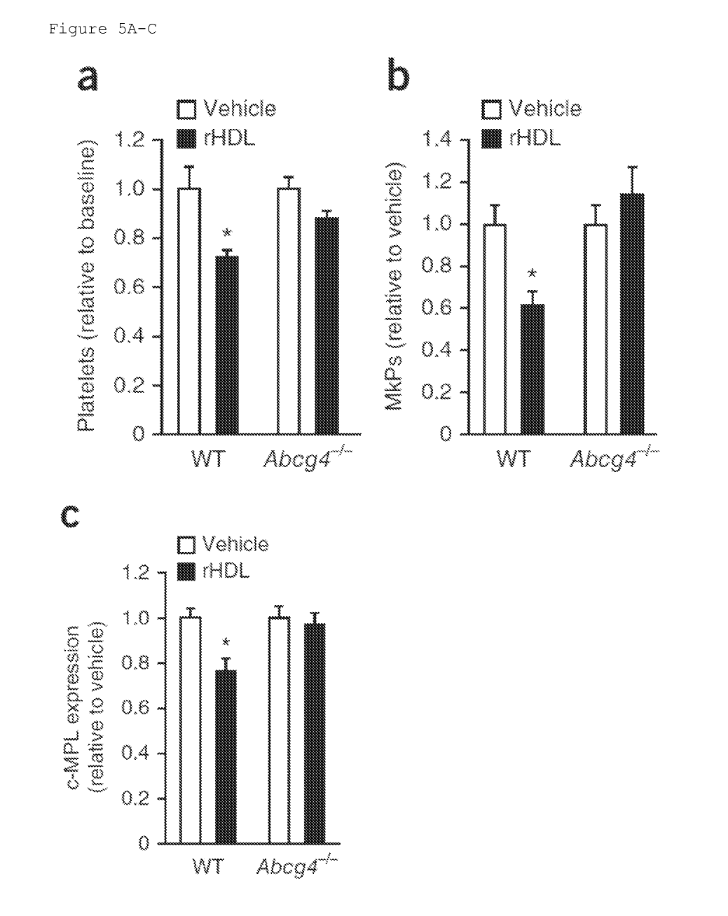

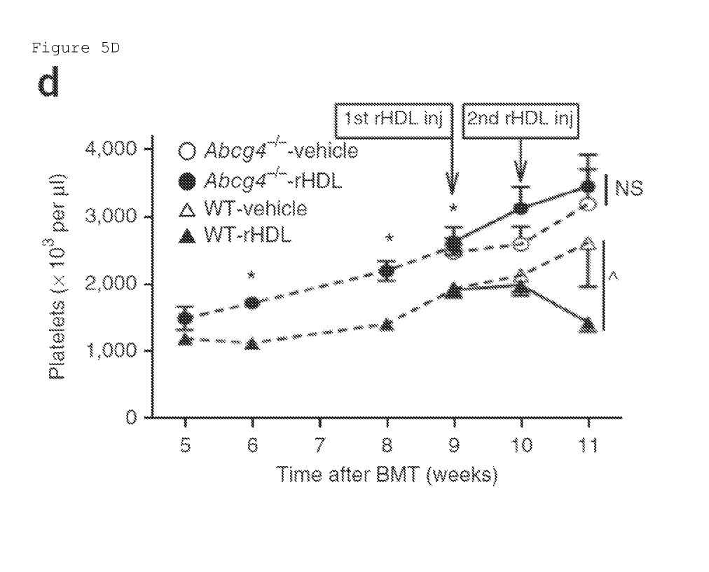

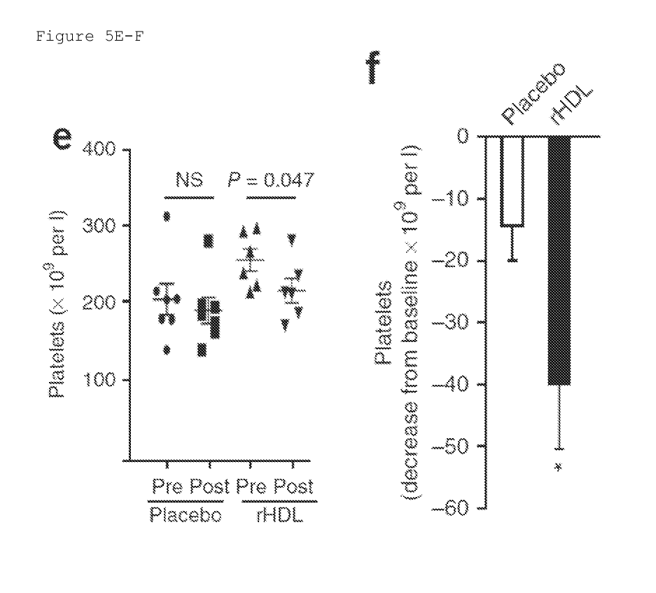

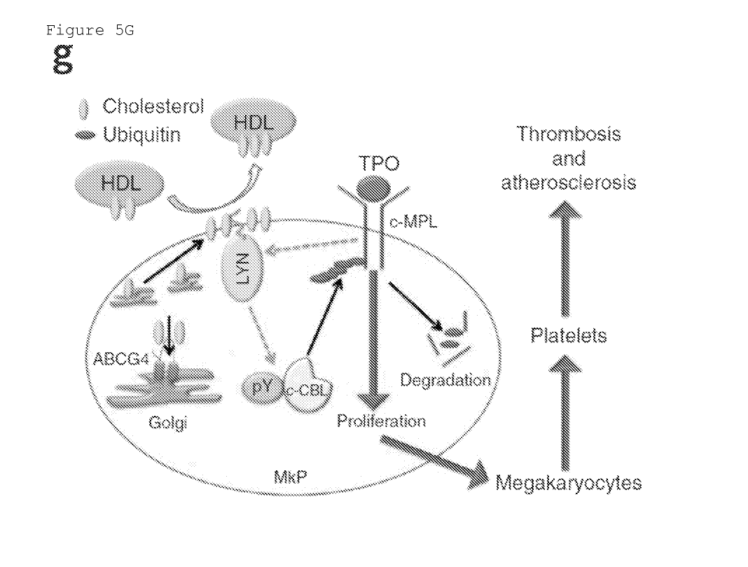

To test whether HDL administration can reduce MkP proliferation and platelet counts in vivo, a preparation of rHDL that has been shown previously to reduce coronary atheroma volume in humans (Tardif, J. C. et al. 2007) was infused into WTD-fed Ldlr−/− mice with or without ABCG4 deficiency. rHDL, but not saline, infusion significantly decreased platelet counts by ˜30% in Ldlr−/− but not Abcg4−/− Ldlr−/− mice (FIG. 5a). HDL infusion also caused decreased c-MPL expression on MkPs, decreased numbers and proliferation of MkPs and decreased megakaryocyte counts in spleen and bone marrow in Abcg4+/+ mice; however, rHDL had no effect in Abcg4−/− mice (FIG. 5b,c and FIG. 15a-c), These findings demonstrate an essential role of ABCG4 in mediating the ability of rHDL to reduce MkP proliferation and platelet counts.

The therapeutic potential for rHDL to reduce platelet counts was explored by testing the effects of rHDL infusion in a mouse model of myelofibrosis and essential thrombocytosis. In this model, mice are transplanted with bone marrow cells transduced with a retrovirus expressing an activating mutant form of c-MPL (c-MPL^{W515L}), found in human myeloproliferative neoplasms (Pikman, Y. et al. 2006; Koppikar, P. et al. 2010). Such c-MPL mutations are found in a subset of patients with myelofibrosis (˜10%) and essential thrombocytosis (˜4-5%) and cause proliferation of MEPs, megakaryocyte expansion and thrombocytosis (Tefferi, A. et al. 2011; Pikman, Y. et al. 2006). The activity of this mutant form of c-MPL requires cell surface localization (Marty, C. et al. 2009). Because cell surface c-MPL expression was increased in Abcg4−/− mice (FIG. 2g), it was hypothesized that c-MPL activity might be enhanced by ABCG4 deficiency. Indeed, thrombocytosis developed more rapidly and was more pronounced in mice transplanted with Abcg4−/− bone marrow cells with reteroviral-mediated expression of c-MPLW515L compared to mice that received WT c-MPL^{W515L} bone marrow cells (FIG. 5d). Although rHDL infusions effectively reversed thrombocytosis in WT c-MPL^{W515L} bone marrow-transplanted mice, the same treatment had no effect on platelet counts in mice transplanted with Abcg4−/− c-MPLW515L bone marrow cells.

To test whether rHDL infusion in humans could reduce platelet numbers, data obtained from a previously reported study involving patients with peripheral vascular disease was analyzed (Shaw, J. A. et al. 2009). This revealed that infusion of rHDL, but not placebo, was associated with a significant reduction of platelet counts (FIG. 5e); when normalized to baseline platelet values, the change was still significantly different between the groups (FIG. 5f). The reduction of platelets in this study was moderate, and platelet counts were maintained in the normal range (FIG. 5e). Together these findings suggest that HDL and ABCG4 promote cholesterol efflux from MkPs and thus facilitate the negative feedback regulation of c-MPL by TPO in MkPs (FIG. 5g).

### Example 10

### Antagonism of ABCG4 Activity

TPO is the most important growth factor regulating megakaryocyte and platelet development and production in vivo (Kaushansky, K. et al. 1998). Platelets are produced by mature megakaryocytes and megakaryocytes are derived from megakaryocyte progenitors (MkP). TPO receptor, c-MPL, is highly expressed in these progenitor cells and megakaryocytes and considered to be essential for megakaryopoiesis and thrombocytopoiesis (Hitchcock, I. S. et al. 2008).

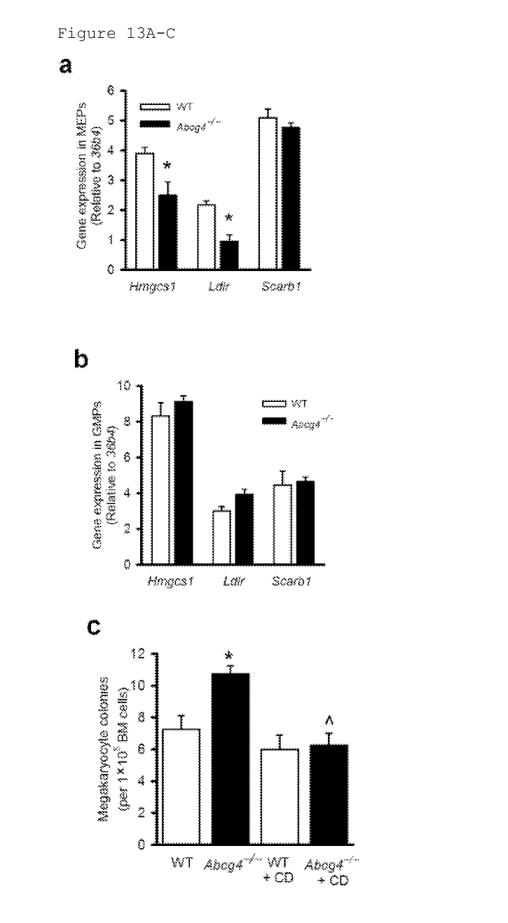

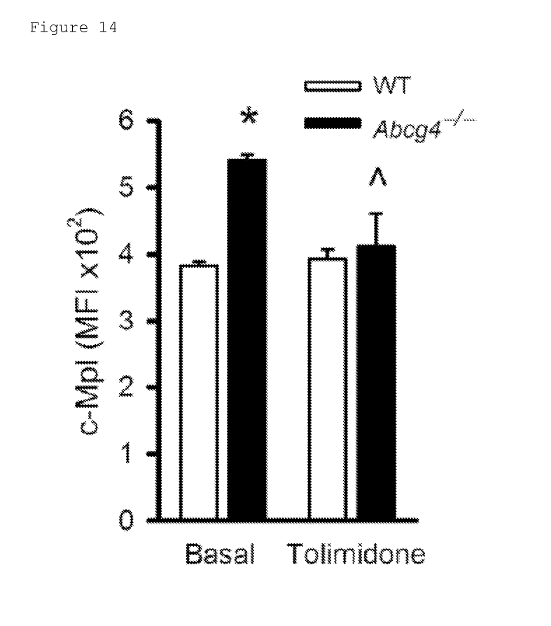

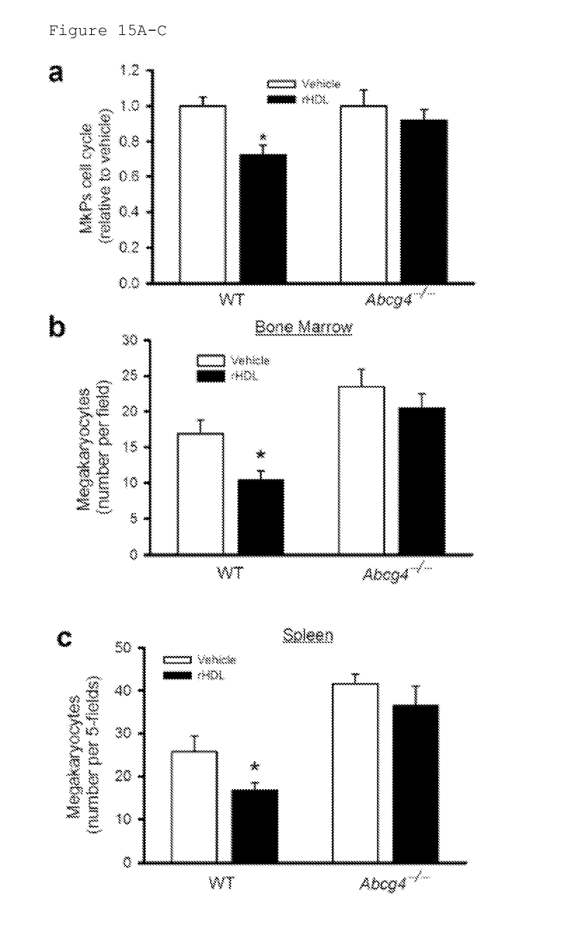

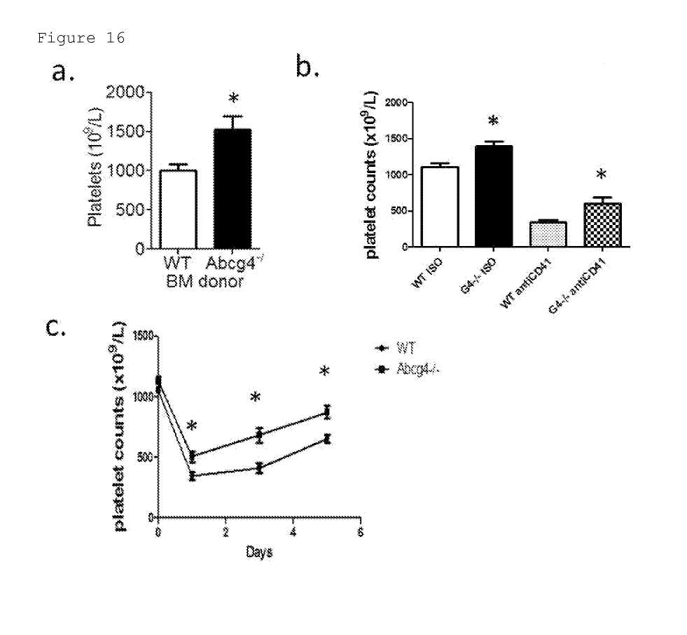

In a mouse model of ITP, it was shown that ABCG4 deficiency causes more rapid recovery of platelet counts, indicating that ABCG4 antagonists could be used as a treatment for thrombocytopenia. It was demonstrated that mice with ABCG4 deletion from bone marrow cells displayed increased platelet count (FIG. 16a). The increased platelet count was due to ABCG4 deficiency in MkPs that resulted in increased MkP proliferation, megakaryocyte production and platelet generation. Importantly, in a mouse ITP model induced by anti-platelet antibody injection, ABCG4 deficiency, in bone marrow (FIG. 16b) or in whole body (FIG. 16c), resulted in more resistance to and/or more rapid recovery from the anti-platelet antibody induced decrease of platelet count. Thus, antagonism or inhibition of ABCG4 activity, such as ABCG4 inhibitors, is useful to treat thrombocytopenia.

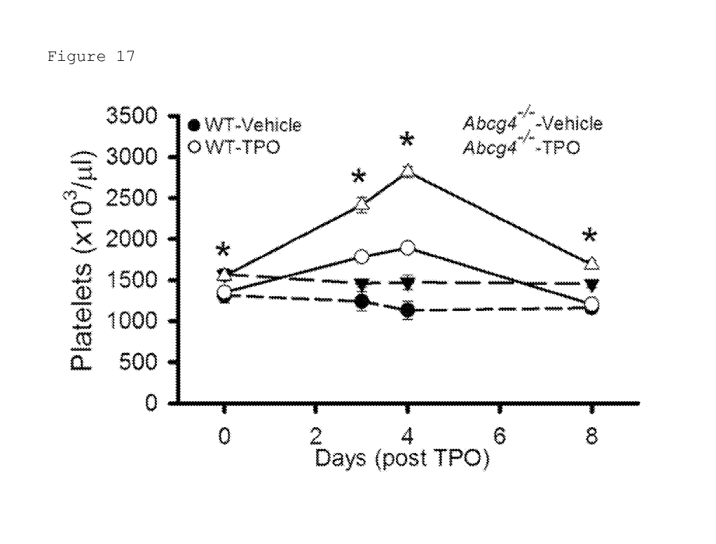

It was also shown that ABCG4 deficiency is associated with markedly increased platelet counts in response to TPO, indicating that ABCG4 antagonism could work synergistically with this agent for treatment of low platelet conditions. TPO injection is known to increase platelet count in mice. A single injection of TPO induced increase of platelet count in the wild type mice (FIG. 17). TPO injection also induced increase of platelet count in Abcg4−/− mice but this increase was much more pronounced as compared with that of the wild type mice (FIG. 17). This indicates that ABCG4 antagonism or inhibition can be used with TPO or its mimetics such as NPlate® to synergistically stimulate platelet production.

### Example 11

### Antagonism of Lyn Kinase and c-CBL Ubiquitin E3 Ligase (c-CBL) Activity

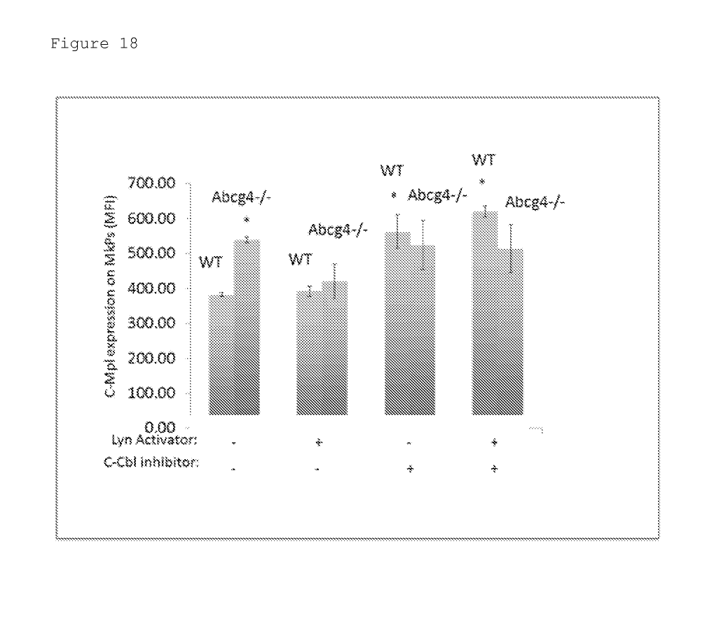

Studies of ABCG4 have indicated downstream signaling by Lyn Kinase and c-CBL, indicating that inhibitors of these signaling molecules could also be used to increase platelet counts. Unlike ABCG4, there exist previous reports indicating that LYN and c-CBL are involved in regulation of platelet production (Huo, Y. et al. 2003; Coller, B. S. 2011). ABCG4 works via modulating LYN kinase and c-CBL ubiquitin E3 ligase activity to regulate platelet production, likely in MkP and other megakaryocyte progenitor cells (FIG. 5g). Indeed, LYN kinase activator decreased cell surface c-MPL levels in MkPs while a c-CBL inhibitor increased them (FIG. 18). Thus, LYN and c-CBL antagonista or inhibitors are useful as treatments of ITP and chemotherapy induced thrombocytopenia.

### Example 12

### Administration of an Antagonist or Inhibitor of ABCG4, LYN or c-CBL

An amount of one or more of an inhibitior or antagonist of ABCG4, Lyn kinase or c-CBL is administered to a subject afflicted with thrombocytopenia. The amount of the inhibitior or antagonist is effective to increase platelet count in the subject.

An amount of an inhibitior or antagonist of ABCG4, an inhibitior or antagonist of Lyn kinase or an inhibitior or antagonist of c-CBL is administered to a subject afflicted with thrombocytopenia. The amount of the inhibitior or antagonist is effective to increase platelet count in the subject.

An amount of two of an inhibitior or antagonist of ABCG4, an inhibitior or antagonist of Lyn kinase or an inhibitior or antagonist of c-CBL is administered to a subject afflicted with thrombocytopenia. The amount of the inhibitior or antagonist is effective to increase platelet count in the subject.

An amount of an inhibitior or antagonist of ABCG4, an inhibitior or antagonist of Lyn kinase and an inhibitior or antagonist of c-CBL is administered to a subject afflicted with thrombocytopenia. The amount of the inhibitior or antagonist is effective to increase platelet count in the subject.

An amount of one or more of an inhibitior or antagonist of ABCG4, LYN or c-CBL is administered to a subject afflicted with thrombocytopenia. An amount of a thrombopoietin mimetic is also administered to the subject. The amount of the inhibitior or antagonist and the thrombopoietin mimetic is effective to increase platelet count in the subject.

An amount of a compound having the structure:

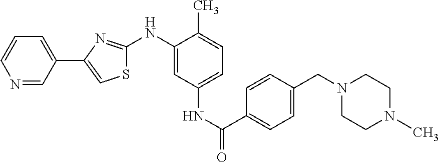 [SMILES: Cc1ccc(NC(=O)c2ccc(CN3CCN(C)CC3)cc2)cc1Nc1nc(-c2cccnc2)cs1]

is administered to a subject afflicted with thrombocytopenia. The amount of the compound is effective to increase platelet count in the subject.

An amount of a compound having the structure:

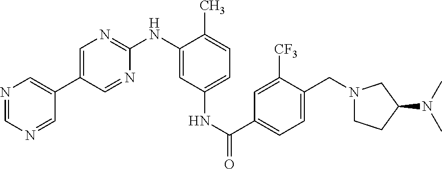 [SMILES: Cc1ccc(NC(=O)c2ccc(CN3CC[C@H](N(C)C)C3)c(C(F)(F)F)c2)cc1Nc1ncc(-c2cncnc2)cn1]

is administered to a subject afflicted with thrombocytopenia. The amount of the compound is effective to increase platelet count in the subject.

An amount of a compound having the structure:

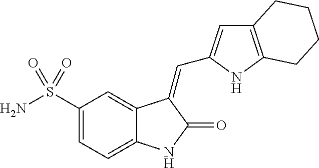 [SMILES: NS(=O)(=O)c1ccc2c(c1)/C(=C/c1cc3c([nH]1)CCCC3)C(=O)N2]

is administered to a subject afflicted with thrombocytopenia. The amount of the compound is effective to increase platelet count in the subject.

An amount of a compound having the structure:

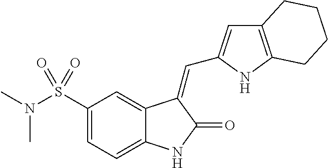 [SMILES: CN(C)S(=O)(=O)c1ccc2c(c1)/C(=C/c1cc3c([nH]1)CCCC3)C(=O)N2]

is administered to a subject afflicted with thrombocytopenia. The amount of the compound is effective to increase platelet count in the subject.

An amount of a compound having the structure:

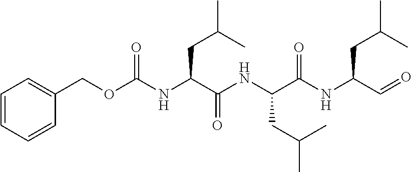 [SMILES: CC(C)C[C@@H](C=O)NC(=O)[C@H](CC(C)C)NC(=O)[C@H](CC(C)C)NC(=O)OCc1ccccc1]

is administered to a subject afflicted with thrombocytopenia. The amount of the compound is effective to increase platelet count in the subject.

An amount of a compound having the structure:

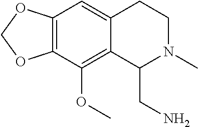 [SMILES: COc1c2c(cc3c1C(CN)N(C)CC3)OCO2]

is administered to a subject afflicted with thrombocytopenia. The amount of the compound is effective to increase platelet count in the subject.

An amount of a compound having the structure:

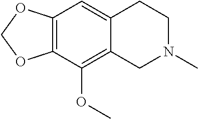 [SMILES: COc1c2c(cc3c1OCO3)CCN(C)C2]

is administered to a subject afflicted with thrombocytopenia. The amount of the compound is effective to increase platelet count in the subject.

An amount of one or more of the compounds having the structure:

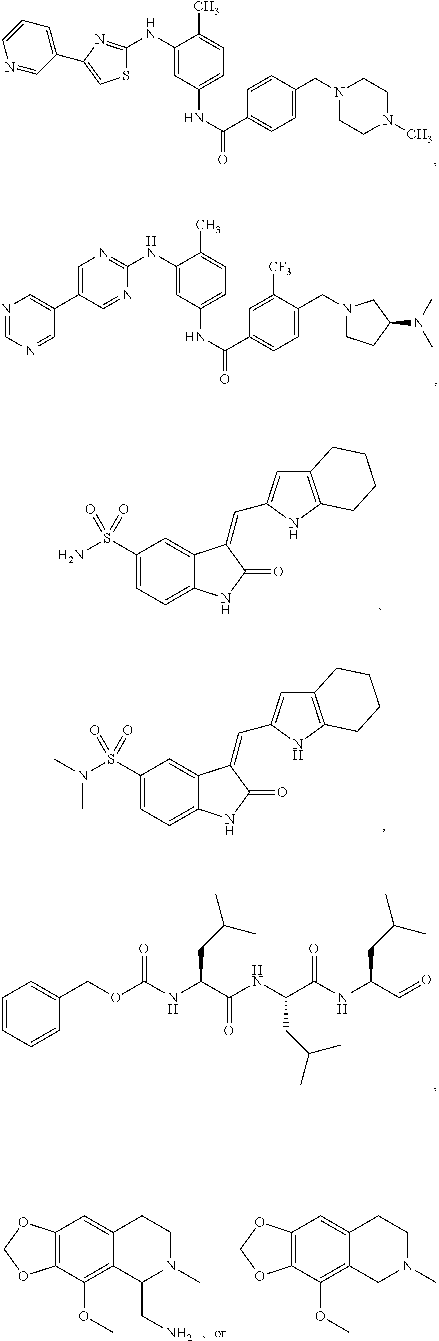

is administered to a subject afflicted with thrombocytopenia. The amount of the compound is effective to increase platelet count in the subject.

### DISCUSSION

Platelets have a key role in atherogenesis and its complications. Both hypercholesterolemia and increased platelet production promote atherothrombosis; however, a potential link between altered cholesterol homeostasis and platelet production has not been explored. Here it is shown that transplantation of bone marrow deficient in ABCG4, a transporter of unknown function, into Ldlr−/− mice resulted in thrombocytosis, accelerated thrombosis and atherosclerosis. Although not detected in atherosclerotic lesions, Abcg4 was highly expressed in bone marrow megakaryocyte progenitors (MkPs). Abcg4−/− MkPs had defective cholesterol efflux to high-density lipoprotein (HDL), increased cell surface expression of the thrombopoietin (TPO) receptor (c-MPL) and enhanced proliferation. These consequences of ABCG4 deficiency seemed to reflect disruption of negative feedback regulation of c-MPL signaling by the E3 ligase c-CBL and the cholesterol-sensing LYN kinase. HDL infusion reduced platelet counts in Ldlr−/− mice and in a mouse model of myeloproliferative neoplasm in an ABCG4-dependent fashion. HDL infusions may offer a new approach to reducing atharothrombotic events associated with increased platelet production.

Atherothrombotic events resulting in heart attack and stroke are the leading cause of morbidity and mortality globally (Labarthe, D. R. et al. 2012). Platelets are involved in multiple steps leading to atherothrombosis, both in the promotion of atherosclerotic plaque growth and also in the formation of thrombi on ruptured or eroded plaques. Increased numbers and activation of platelets both contribute to atherothrombotic risk (Martin, J. F., et al. 2012; Trip, M. D. et al. 1990), and increased platelet production may underlie these processes (Martin, J. F. et al. 2012; Hasselbalch, H. C. et al. 2012). A striking example of increased platelet production occurs in myeloproliferative neoplasms such as myelofibrosis and essential thrombocytosis, in which mutations in the gene encoding c-MPL or in the genes encoding its downstream signaling elements lead to excessive production of megakaryocytes and thrombocytosis (Tefferi, A. et al. 2011). More generally, increased platelet production, denoted by increased platelet volume and increased numbers of circulating reticulated platelets, is a major risk factor for atherosclerotic cardiovascular disease and may precipitate acute coronary syndromes (Martin, J. F. et al. 2012).

Increased amounts of low-density lipoprotein and decreased amounts of HDL are also well known major risk factors for atherothrombosis (Steinberf, D. 2008). The atheroprotective functions of HDL are thought to be mediated by its ability to promote cholesterol efflux from cells in the arterial wall in a process that is facilitated by the ATP-binding cassette transporters ABCA1 and ABCG1 (Tall, A. R. et al. 2008). Although hypercholesterolemia has been associated with increased platelet production, the underlying mechanisms are unclear (Pathansali, R. et al. 2001). Moreover, potential mechanisms linking defective cholesterol efflux pathways to platelet production have not been explored.

The ATP-binding cassette transporter ABCG4, which is highly homologous to ABCG1, promotes, cholesterol efflux to HDL when overexpressed in cultured cells (Wang, N. et. al. 2004; Wang, N. et al. 2006). However ABCG4 is not expressed in macrophage foam cells, and its in vivo function and potential effects on atherogenesis remain unknown. Abcg4 expression has been detected in the brain and hematopoietic tissues such as fetal liver and bone marrow Annilo, T. et al. 2001; Bojanic, D. D. et al. 2010). To uncover how ABCG4 might act in the hematopoietic system, the effects of ABCG4 deficiency on hematopoietic function and atherogenesis in a hypercholesterolemic mouse model of atherosclerosis were assessed.

The studies disclosed herein show that defective cholesterol homeostasis in megakaryocyte progenitor cells promotes megakaryocyte formation, platelet overproduction, arterial thrombosis and atherogenesis. Increased membrane cholesterol concentrations in megakaryocyte progenitors lead to increased amounts and signaling of the TPO receptor. ABCG4 is highly expressed in MkPs, and its deficiency leads to cholesterol accumulation, MkP proliferation and increased platelet production. The ability of rHDL to suppress MEP and MkP proliferation and platelet counts in vivo was dependent on ABCG4, probably reflecting the cell type-restricted pattern of expression of cholesterol efflux-promoting ABC transporters. Therapeutic interventions such as rHDL infusions have the potential to reverse excessive megakaryocytopoiesis in states of platelet overproduction, such as those that occur in myeloproliferative neoplasms.

The idea that cellular sterol metabolism is intimately connected to proliferative responses is longstanding (Pikman, Y. et al. 2006). The requirement for new membrane synthesis during cell proliferation leads to activation of cholesterol biosynthesis involving cleavage of sterol regulatory element binding transcription factor 2 (SREBP-2) and transcriptional induction of cholesterol biosynthetic genes (Brown, M. S. et al. 1974). Recent studies have linked control of cell proliferation to cholesterol efflux pathways mediated by ABCA1, ABCG1 or both (Yvan-Charvet, L. et al. 2010; Murphy, A. J. et al. 2011; Bensinger, S. J. et al. 2008; Armstroing, A. J. et al. 2010). However, specific molecular mechanisms linking cellular cholesterol accumulation to altered growth factor receptor signaling have not been defined. Our studies suggest that LYN kinase may act as a membrane cholesterol sensor, acting upstream of c-CBL to modulate its downregulation of c-MPL. This hypothesis is supported by previous studies showing that LYN kinase activity is modulated by altered membrane cholesterol concentrations (Oneyama, C. et al. 2009). LYN is palmitoylated, and palmitoylation-defective LYN shows decreased association with cholesterol-rich membranes but an increased ability to mediate tyrosine phosphorylation of immunoglobulin receptors (Kovarova, M. et al. 2001). It was shown that infusions of cholesterol-poor rHDL were associated with a reduction in platelet counts in a previous small study involving patients undergoing treatment for peripheral vascular disease, suggesting the potential human relevance of our findings. Moreover, in a recent human genome-wide association study, SNPs in or near the c-CBL (also called CBL) gene were associated with platelet counts (Gieger, C. et al. 2011). Interestingly, ABCG4 is in tight linkage disequilibrium with c-CBL, and SNPs associated with platelet counts could be influencing expression of c-CBL and/or ABCG4 (Gieger, C. et al. 2011). Our findings suggest a potential mechanism linking expression of ABCG4 to the regulation of platelet counts involving defective c-CBL-mediated feedback regulation of c-MPL and thus support the concept that these genes act in megakaryocytes or their progenitors to regulate platelet production (Gieger, C. et al. 2011).

There is tremendous interest in the development of new therapies that increase plasma HDL concentrations as potential treatments for atherosclerotic cardiovascular disease. The achievement of this goal has been challenging, as highlighted by the recent failure of treatments that increase HDL concentrations in clinical trials, such as the CETP inhibitors torcetrapib and dalcetrapib or ER niacin. However, approaches that actively increase the flux of cholesterol from macrophages and other cells remain promising treatments to reduce coronary atherosclerosis (Tardif, J. C. et al. 2007; Rader, D. J. et al. 2012). The studies disclosed herein suggest that such treatments may have the beneficial effects of suppressing MEP and MkP proliferation.

Thrombocytosis in essential thrombocytosis and myelofibrosis is currently treated with low-dose aspirin, and high-risk patients with essential thrombocytosis (>60 years old or having experienced a previous thrombotic event) are treated with genotoxic agents such as hydroxyurea (Verstovsek, S. et al. 2010). There remains a need for new therapies for patients with myelofibrosis given their poor overall outcome and limited therapeutic options (Wolanskyj, A. P. et al. 2006). The studies disclosed herein suggest that rHDL infusion may specifically reverse c-MPL-dependent MEP proliferation and aberrant megakaryopoiesis underlying thrombocytosis in essential thrombocytosis and myelofibrosis. Moreover, increased platelet production is a cardiovascular risk factor and has been implicated more generally in the precipitation of atherothrombotic events6. Thus, rHDL infusions could complement existing treatments that directly target platelets or clotting factors. rHDL infusions may have multiple beneficial effects in the setting of acute coronary syndromes, including the removal of cholesterol, the suppression of inflammation in plaques and the suppression of excessive myeloid cell production and extramedullary hematopoiesis, as well as limiting the overproduction of platelets (Dutta, P. et al. 2012; Tall, A. R. et al. 2012). rHDL preparation and infusion as a chronic therapy remains challenging.

A novel mechanism has been identified which indicates that inhibition of ABCG4, Lyn kinase or C-CBL activity increases platelet count and thus is useful for the treatment of thrombocytopenia. The findings disclosed herein show that ABCG4 works via modulating Lyn kinase and c-CBL ubiquitin E3 ligase (c-CBL) activity to regulate platelet production. Therefore, antagonism or inhibition of one or more of ABCG4, Lyn kinase or c-CBL increases platelet production and platelet count.

### REFERENCES

Annilo, T. et al. Human and mouse orthologs of a new ATP-binding cassette gene, ABCG4. Cytogenet. Cell Genet. 94, 196-201 (2001).
Armstrong, A. J., Gebre, A. K., Parks, J. S. & Hedrick, C. C. ATP-binding cassette transporter G1 negatively regulates thymocyte and peripheral lymphocyte proliferation. J. Immunol. 184, 173-183 (2010).
Barter, P. J. et al. Effects of torcetrapib in patients at high risk for coronary events. N. Engl. J. Med. 357, 2109-2122 (2007).
Bensinger, S. J. et al. LXR signaling couples sterol metabolism to proliferation in the acquired immune response. Cell 134, 97-111 (2008).
Bersenev, A., Wu, C., Balcerek, J. & Tong, W. Lnk controls mouse hematopoietic stem cell self-renewal and quiescence through direct interactions with JAK2. J. Clin. Invest. 118, 2832-2844 (2008).
Blake, R. A. et al. SU6656, a selective src family kinase inhibitor, used to probe growth factor signaling. Mol. Cell Biol. 20. 9018-9027 (2000),
Bojanic, D. D. et al. Differential expression and function of ABCG1 and ABCG4 during development and aging. J. Lipid Res. 51, 169-181 (2010).
Brown, M. S. & Goldstein, J. L. Suppression of 3-hydroxy-3-methylglutaryl coenzyme A reductase activity and inhibition of growth of human fibroblasts by 7-ketocholesterol. J. Biol. Chem. 249, 7306-7314 (1974).
Brown, M. S. & Goldstein, J. L. Multivalent feedback regulation of HMG CoA reductase, a control mechanism coordinating isoprenoid synthesis and cell growth. J. Lipid Res. 21, 505-517 (1980).
Brown, M. S. & Goldstein, J. L. Cholesterol feedback: from Schoenheimer's bottle to Scap's MELADL. J. Lipid Res. 50 (suppl.), S15-S27 (2009).
Chan, V. W., Meng, F., Soriano, P., DeFranco, A. L. & Lowell, C. A. Characterization of the B lymphocyte populations in Lyn-deficient mice and the role of Lyn in signal initiation and down-regulation. Immunity 7, 69-81 (1997).
Coller, B. S. Historical perspective and future directions in platelet research. J. Thromb. Haemost. 9 (suppl. 1), 374-395 (2011).
Dubreuil, P. et al. Masitinib (AB1010), a Potent and Selective Tyrosine Kinase Inhibitor Targeting KIT. Volume 4, Issue 9, p. e7258 (2009).
Dutta, P. et al. Myocardial infarction accelerates atherosclerosis. Nature 487, 325-329 (2012).
Erhurt, M. A, et. al. Thrombocytopenia in Adults: Review Article, J hematol, I (2-3), p. 44-53 (2012).
Frontelo, P. et al. Novel role for EKLF in megakaryocyte lineage commitment. Blood 110, 3871-3880 (2007).
Gieger, C. et al. New gene functions in megakaryopoiesis and platelet formation. Nature 480, 201-208 (2011).
Guthikonda, S. et al. Role of reticulated platelets and platelet size heterogeneity on platelet activity after dual antiplatelet therapy with aspirin and clopidogrel in patients with stable coronary artery disease. J. Am. Coll. Cardiol. 52, 743-749 (2008).
Hasselbalch, H. C. Perspectives on chronic inflammation in essential thrombocythemia, polycythemia vera, and myelofibrosis. Blood 3219-3225 (2012).
Hitchcock, I. S., Chen, M. M., King, J. R. & Kaushansky, K. YRRL motifs in the cytoplasmic domain of the thrombopoietin receptor regulate receptor internalization and degradation. Blood 112, 2222-2231 (2008).
Hunter, S., Burton, E. A., Wu, S. C. & Anderson, S. M. Fyn associates with Cbl and phosphorylates tyrosine 731 in Cbl, a binding site for phosphatidylinositol 3-kinase. J. Biol. Chem. 274, 2097-2106 (1999).
Huo, Y. et al. Circulating activated platelets exacerbate atherosclerosis in mice deficient in apolipoprotein E. Nat. Med. 9, 61-67 (2003).
Imbach, P. & Crowther, M. Thrombopoietin receptor agonists for primary immune thrombocytopenia. The New England journal of medicine 365, 734-741 (2011).
Ingley, E. et al. Lyn deficiency reduces GATA-1, EKLF and STAT5, and induces extramedullary stress erythropoiesis. Oncogene 24, 336-343 (2005).
Jenkins, J. et al. Phase 1 clinical study of eltrombepag, an oral, nonpeptide thrombopoietin receptor agonist. Blood Jun. 1, 2007 vol. 109 no. 11 4739-4741.
Kaushansky, K. Thrombopoietin. The New England journal of medicine 339, 746-754 (1998).
Kelemen, E., Lehoczky, D., Jakab, K., Batai, A. & Vargha, P. Responses to single-dose thrombopoietin decrease with higher platelet counts in mice. Acta Haematol. 101, 41-45 (1999).
Koenen, R. R. et al. Disrupting functional interactions between platelet chemokines inhibits atherosclerosis in hyperlipidemic mice. Nat. Med. 15, 97-103 (2009).
Koppikar, P. at al. Efficacy of the JAK2 inhibitor INCB16562 in a murine model of MPLW515L-induced thrombocytosis and myelofibrosis. Blood 115, 2919-2927 (2010).
Kovárová, M. et al. Structure-function analysis of Lyn kinase association with lipid rafts and initiation of early signaling events after Fcε receptor I aggregation. Mol. Cell Biol. 21, 8318-8328 (2001).
Kuter, D. J. et al. Romiplostim or Standard of Care in Patients with Immune Thrombocytopenia N Engl J Med 2010; 363:1889-1899 (2010).
Labarthe, D. R. & Dunbar, S. B. Global cardiovascular health promotion and disease prevention: 2011 and beyond. Circulation 125, 2667-2676 (2012).
Lakkis, N. et al. Reticulated platelets in acute coronary syndrome: a marker of platelet activity. J. Am. Coll. Cardiol. 44, 2091-2093 (2004).
Lannutti, B. J., Shim, N. H., Blake, N., Reems, J. A. & Drachman, J. G. Identification and activation of Src family kinases in primary megakaryocytes. Exp. Hematol. 31, 1268-1274 (2001).
Lannutti, B. J., Minear, J., Blake, N. & Drachman, J. G. Increased megakaryocytopoiesis in Lyn-deficient mice. Oncogene 25, 3316-3324 (2006).
Libby, P., Ridker, P. M. & Hansson, G. K. Progress and challenges in translating the biology of atherosclerosis. Nature 473, 317-325 (2011).
Lingwood, D. & Simons, K. Lipid rafts as a membrane-organizing principle. Science 327, 46-50 (2010).
Mause, S. F., von Hundelshausen, P., Zernecke, A., Koenen, R. R. & Weber, C. Platelet microparticles: a transcellular delivery system for RANTES promoting monocyte recruitment on endothelium. Arterioscler. Thromb. Vasc. Biol. 25, 1512-1518 (2005).
Martin, J. F., Kristensen, S. D., Mathur, A., Grove, E. L. & Choudry, F. A. The causal role of megakaryocyte-platelet hyperactivity in acute coronary syndromes. Nat. Rev. Cardiol. 9, 658-670 (2012).
Marty, C. et al. Ligand-independent thrombopoietin mutant receptor requires cell surface localization for endogenous activity. J. Biol. Chem. 284, 11781-11791 (2009).
Mazzone, A. et al. Increased expression of neutrophil and monocyte adhesion molecules in unstable coronary artery disease. Circulation 88, 358-363 (1993).
Meurs, I. et al. The effect of ABCG1 deficiency on atherosclerotic lesion development in LDL receptor knockout mice depends on the stage of atherogenesis. Atherosclerosis 221, 41-47 (2012).
Murphy, A. J. et al. ApoE regulates hematopoietic stem cell proliferation, monocytosis, and monocyte accumulation in atherosclerotic lesions in mice. J. Clin, Invest. 121, 4138-4149 (2011).
Nadeau, S. et al. Oncogenic signaling by leukemia-associated mutant Cbl proteins. Biochem. Anal. Biochem. S6 (2012).
Nofer, J. R. & van Eck, M. HDL scavenger receptor class B type I and platelet function. Curr. Opin. Lipidol. 22, 277-282 (2011).
Oneyama, C. et al. Transforming potential of Src family kinases is limited by the cholesterol-enriched membrane microdomain. Mol. Cell Biol. 29, 6462-6472 (2009).
Pathansali, R., Smith, N. & Bath, P. Altered megakaryocyte-platelet haemostatic axis in hypercholesterolaemia. Platelets 12, 292-297 (2001).
Pikman, Y. et al. MPLW515L is a novel somatic activating mutation in myelofibrosis with myeloid metaplasia. PLoS Med. 3, e270 (2006).
Rader, D. J. & Tall, A. R. The not-so-simple HDL story: is it time to revise the HDL cholesterol hypothesis? Nat. Med. 18, 1344-1346 (2012).
Ranalletta, M. et al. Decreased atherosclerosis in low-density lipoprotein receptor knockout mice transplanted with Abcg1−/− bone marrow. Arterioscler. Thromb. Vasc. Biol. 26, 2308-2315 (2006).
Santos, F. P. et al. Bafetinib, a dual Bcr-Abl/Lyn tyrosine kinase inhibitor for the potential treatment of leukemia. Curr Opin Investig Drugs. 11(12), 1450-65 (2010).
Saporito, M. S., Ochman, A. R., Lipinski, C. A., Handler, J. A. & Reaume, A. G. MLR-1023 a potent and selective allosteric activator of Lyn kinase in vitro that improves glucose tolerance in vivo. J. Pharmacol. Exp. Ther. 342-22 2012).
Saur, S. J., Sangkhae, V., Geddis, A. E., Kaushansky, K. & Hitchcock, I. S. Ubiquitination and degradation of the thrombopoietin receptor c-Mpl. Blood 115, 1254-1263 (2010).
Shaw, J. A. et al. Infusion of reconstituted high-density lipoprotein leads to acute changes in human atherosclerotic plaque. Circ. Res. 103, 1084-1091 (2008).
Steinberg, D. The statins in preventive cardiology. N. Engl. J. Med. 359, 1426-1427 (2008).
Stohlawetz, P. et al. Measurement of the levels of reticulated platelets after plateletpheresis to monitor activity of thrombopoiesis. Transfusion 38, 454-458 (1998).
Tall, A. R., Yvan-Charvet, L., Terasaka, N., Pagler, T. & Wang, N. HDL, ABC transporters, and cholesterol efflux: implications for the treatment of atherosclerosis. Cell Metab. 7, 365-375 (2008).
Tall, A. R., Yvan-Charvet, L., Westerterp, M. & Murphy, A. J. Cholesterol efflux: a novel regulator of myelopoiesis and atherogenesis. Arterioscler. Thromb. Vasc. Biol. 32, 2547-2552 (2012).
Tardif, J. C. et al. Effects of reconstituted high-density lipoprotein infusions on coronary atherosclerosis: a randomized controlled trial. J. Am. Med. Assoc. 297, 1675-1682 (2007).
Tefferi, A. & Vainchenker, W. Myeloproliferative neoplasms: molecular pathophysiology, essential clinical understanding, and treatment strategies. J. Clin. Oncol. 29, 573-582 (2011).
Tiedt, R. et al. Pronounced thrombocytosis in transgenic mice excressinc reduced levels of Mpl in platelets and terminally differentiated megakaryocytes. Blood 113, 1768-1777 (2009).
Tong, W., Ibarra, Y. M. & Lodish, H. F. Signals emanating from the membrane proximal region of the thrombopoietin receptor (mpl) support hematopoietic stem cell self-renewal. Exp. Hematol. 35, 1447-1455 (2007).
Trip, M. D., Cats, V. M., van Capelle, F. J. & Vreeken, J. Platelet hyperreactivity and prognosis in survivors of myocardial infarction. N. Engl. J. Med. 322, 1549-1554 (1990).
Verstovsek, S. et al. Safety and efficacy of INCB018424, a JAK1 and JAK2 inhibitor, in myelofibrosis. N. Engl. J. Med. 363, 1117-1127 (2010).
Villmow, T., Kemkes-Matthes, B. & Matzdorff, A. C. Markers of platelet activation and platelet-leukocyte interaction in patients with myeloproliferative syndromes. Thromb. Res. 108, 139-145 (2002).
Wang, N., Lan, D., Chen, W., Matsuura, F. & Tall, A. R. ATP-binding cassette transporters G1 and G4 mediate cellular cholesterol efflux to high-density lipoproteins. Proc. Natl. Acad. Sci. USA 101, 9774-9779 (2004).
Wang, N., Ranalletta, M., Matsuura, F., Peng, F. & Tall, A. R. LXR-induced redistribution of ABCG1 to plasma membrane in macrophages enhances cholesterol mass efflux to HDL. Arterioscler. Thromb. Vasc. Biol. 26, 1310-1316 (2006).
Wolanskyj, A. P., Schwager, S. M., McClure, R. F., Larson, D. R. & Tefferi, A. Essential thrombocythemia beyond the first decade: life expectancy, long-term complication rates, and prognostic factors. Mayo Clin. Proc. 81, 159-166 (2006).
Yvan-Charvet, L. et al. ATP-binding cassette transporters and HDL suppress hematopoietic stem cell proliferation. Science 328, 1689-1693 (2010).

1. A method of treating a subject to increase the subject's platelet count which comprises administering to the subject an amount of one of more of an antagonist or inhibitor of ABCG4, Lyn kinase or c-CBL effective to antagonize or inhibit such ABCG4, Lyn kinase or c-CBL so as to thereby increase the subject's platelet count.

2. The method of claim 1, wherein one of an antagonist or inhibitor of ABCG4, an antagonist or inhibitor of Lyn kinase or an antagonist or inhibitor of c-CBL is administered.

3. The method of claim 2, wherein an antagonist or inhibitor of ABCG4 is administered.

4. The method of claim 2, wherein an antagonist or inhibitor of Lyn kinase is administered.

5. The method of claim 2, wherein an antagonist or inhibitor of c-CBL is administered.

6. The method of claim 1, wherein two of an antagonist or inhibitor ABCG4, an antagonist or inhibitor of Lyn kinase or an antagonist or inhibitor of c-CLBL is administered.

7. The method of claim 6, wherein an antagonist or inhibitor of ABCG4 and an antagonist or inhibitor of Lyn kinase is administered.

8. The method of claim 6, wherein an antagonist or inhibitor of ABCG4 and an antagonist or inhibitor of c-CBL is administered.

9. The method of claim 6, wherein an antagonist or inhibitor of Lyn kinase and an antagonist or inhibitor of c-CBL is administered.

10. The method of claim 1, wherein an antagonist or inhibitor of ABCG4, an antagonist or inhibitor of Lyn kinase and an antagonist or inhibitor of c-CBL is administered.

11. The method of claim 1, wherein the Lyn kinase antagonist or inhibitor has the structure:

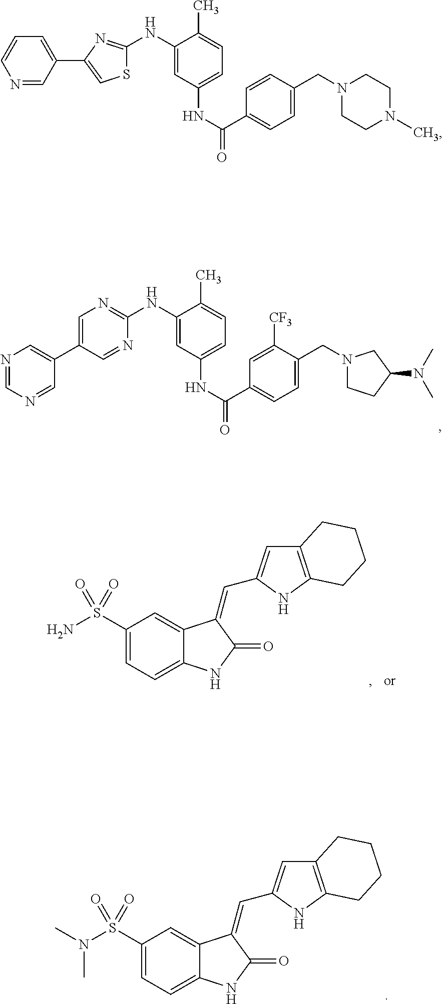

12. The method of claim 1, wherein the c-CBL antagonist or inhibitor has the structure:

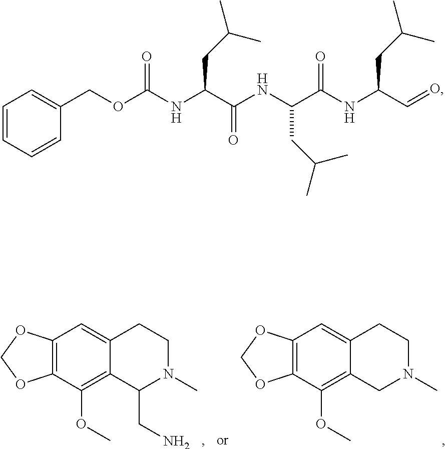 [SMILES: CC(C)C[C@@H](C=O)NC(=O)[C@H](CC(C)C)NC(=O)[C@H](CC(C)C)NC(=O)OCc1ccccc1 ; COc1c2c(cc3c1C(CN)N(C)CC3)OCO2 ; COc1c2c(cc3c1OCO3)CCN(C)C2]

13. The method of claim 1, which further comprises administering to the subject thrombopoietin or a thrombopoietin mimetic.

14. (canceled)

15. The method of claim 13, wherein the thrombopoietin mimetic is romiplostim or eltrombopag.

16. (canceled)

17. The method of claim 1, wherein megakaryocyte production in the subject is increased.

18. The method of claim 1, wherein the proliferation of megakaryocyte progenitor cells in the subject is increased.

19. The method of claim 1, wherein platelet production in the subject is increased.

20. The method of claim 1, wherein the subject is suffering from thrombocytopenia.

21. The method of claim 20, wherein the thrombocytopenia is idiopathic immune thrombocytopenia purpura.

22. The method of claim 20, wherein the thrombocytopenia is immune thrombocytopenia purpura.

23. The method of claim 20, wherein the thrombocytopenia is chemotherapy-induced thrombocytopenia.

24. The method of claim 20, wherein the thrombocytopenia is drug-induced thrombocytopenia.

25. (canceled)
